(19) United States
(12) Patent Application Publication
(10) Pub. No.: US 2011/0104736 A1
(43) Pub. Date: May 5, 2011

(54) SELECTION OF ORGANISMS CAPABLE OF FERMENTING MIXED SUBSTRATES

(76) Inventors: Jacobus Thomas Pronk, Schipluiden (NL); Antonius Antonius Maris Van, Delft (NL); Hendrik Wouter Wisselink, Culemborg (NL)

(21) Appl. No.: 12/921,937

(22) PCT Filed: Mar. 10, 2009
(86) PCT No.: PCT/EP2009/052754

§ 371 Date: Dec. 15, 2010

(30) Foreign Application Priority Data
Mar. 13, 2008 (EP) 08102590.0

(51) Int. Cl. C12Q 1/02; C12N 1/14; C12N 1/16; C12P 1/02; C12P 7/06; C12P 7/16; C12P 7/56; C12P 7/42; C12P 7/40; C12P 7/54; C12P 7/46; C12P 7/48; C12P 13/00; C12P 7/18; C12P 5/02; C12P 7/20; C12P 35/00; C12P 37/00

(52) U.S. Cl. 435/29; 435/254.1; 435/255.1; 435/255.2; 435/171; 435/161; 435/160; 435/139; 435/146; 435/136; 435/140; 435/145; 435/144; 435/128; 435/158; 435/167; 435/159; 435/47; 435/43

(57) ABSTRACT

The present invention relates to a method for selecting a strain of an organism capable of improved consumption of a mixed substrate comprising two or more carbon sources as compared to a reference strain of the organism, which method comprises: growing a population of the reference strain of the organism in the presence of the two or more carbon sources, wherein the number of generations of growth of the said population on each of the said carbon sources is at least about 50% of the number of generations of growth on the carbon source most preferred by the organism; and selecting the resulting strain of the organism, thereby to select a strain of the organism capable of improved consumption of a mixed substrate comprising the two or more carbon sources as compared to the reference strain of the organism. The invention also relates to strains of organisms selected using such a method. Strains of organisms identified using the selection method may be used in fermentation processes in which a mixed substrate is used.

### FIELD OF THE INVENTION

The present invention relates to a method for selecting strains of an organism which are capable of improved consumption of mixted of substrates. The invention also relates to strains of organisms which have been selected by such a process and to the use of strains of organisms identified by the selection method in fermentation processes.

### BACKGROUND OF THE INVENTION

Lignocellulosic feed stocks such as corn stover, wood waste, sugar cane bagasse, are examples of large, but largely untapped renewable carbon sources. The predominant polymer in many renewable feedstocks is cellulose, which generates glucose upon hydrolysis. However, depending on the feedstock in question, large fractions of other six and five carbon sources are released when hemicellulose and pectin are hydrolyzed, for example xylose and arabinose. This necessitates a mixed substrate fermentation.

Mixed sugar fermentations are more complex than standard pure substrate processes. Regulatory events such as transport competition or inhibition, induction, repression and catabolite inactivation can increase fermentation times due to diauxic growth and lag and reduce product yields from the secondary substrates. However and contrastingly, most fermentation research has focused on optimizing product formation from single substrates.

There is thus a need to develop methods whereby product formation from multiple substrates may be optimized. Potential fermentation methods to accelerate simultaneous or sequential mixed substrate utilization and generate high product yields may include: environmental manipulation (e.g., pH, media composition, substrate ratios); pre-induction before large scale fermentations; identification and feeding of metabolic inducers; novel reactor configurations, such as a two-phase fed batch processes (e.g., aerobic growth on the inducer at low concentrations and generation of high cell densities followed by controlled feeding of the mixed sugars for product formation); and use of microorganisms which are specifically adapted for growth on mixed substrates.

### SUMMARY OF THE INVENTION

This invention is based on the development of a selection method which enables a strain of an organism to be selected which can grow efficiently, in particularly more efficiently as compared to a reference strain, on a mixed substrate, i.e. a substrate which comprises two or more carbon sources. The method may, in particular, be used to select a strain of an organism capable of improved growth on such a substrate, i.e. a strain which shows improved/faster consumption of such a substrate. That is to say, the invention may be used to select an improved strain of an organism which is already able to utilize a mixed substrate, but only at a lower rate.

The starting strain of an organism subjected to the selection method of the invention may herein be referred to, for example, as a “starting” strain, a “reference” strain or an “initial” strain or the like.

In the method, a starting (or reference) population of the organism is selected or constructed for growth on the mixed substrate. That starting population is then subjected to the selection method of the invention. The selection method is carried out such that the number of generations of growth of the population of the organism on each of the carbon sources in the mixed substrate is at least about 50% of the number of generations of growth of the population of the organism on the most preferred carbon source.

The selection method described herein has allowed the identification of a strain of yeast which shows improved consumption when grown on a mixed substrate comprising glucose, xylose and arabinose.

According to the invention, there is thus provided a method for selecting a strain of an organism capable of improved/faster consumption of a mixed substrate comprising two or more carbon sources than a reference strain of the organisms, which method comprises:
growing a population of the reference strain of the organism in the presence of the two or more carbon sources, wherein the number of generations of growth of the said population on each of the said carbon sources is at least about 50% of the number of generations of growth on the carbon source most preferred by the reference strain of the organism; and
selecting the resulting strain of the organism,
thereby to select a strain of the organism capable of improved consumption of a mixed substrate comprising the two or more carbon sources as compared to the reference strain of the organism.

The invention also provides:
a strain of an organism identified according to the method of any one of the preceding claims;
a yeast strain, such as a Saccharomyces cerevisiae strain, capable of a specific consumption rate of arabinose of at least about 0.4 g h⁻¹ (g dry weight)⁻¹ and of xylose of at least about 0.2 g h⁻¹ (g dry weight)⁻¹;
a yeast strain, such as a Saccharomyces cerevisiae strain, capable of fermenting a substrate comprising xylose and arabinose, and optionally glucose, giving rise to an ethanol yield of at least about 0.4 g g⁻¹;
a Saccharomyces cerevisiae strain deposited at the Centraalbureau voor Schimmelcultures under the accession number CBS 122701;
a process for producing a fermentation product which process comprises fermenting a substrate containing two or more sources of carbon with a strain of an organism as described above such that the cell ferments the said carbon sources to the fermentation product;
a process for producing a fermentation product which process comprises:
   selecting a strain of an organism capable of consumption of a mixed substrate comprising two or more carbon sources using a method according to the invention; and
   fermenting a medium containing the two or more carbon sources on which the strain of the organism was selected with the strain of the organism thus selected such that the strain of the organism ferments the two or more carbon sources to the fermentation product.

### DESCRIPTION OF THE FIGURES

FIG. 1 shows the CO₂ production profile and residual sugar concentrations during a selective chemostat cultivation of engineered xylose and arabinose utilizing S. cerevisiae cells. CO₂ production profile (solid line); xylose (▪); arabinose ().

FIG. 2 showns anaerobic batch cultivations in MY containing a mixture of 30 g l⁻¹ glucose, 15 g l⁻¹ D-xylose, and 15 g l⁻¹ L-arabinose of strains IMS0003 (A), IMS0007 (B), a 100 mL sample of SBR I (C), and strain IMS0010 (D). Solid line, CO₂ production profile; glucose (); xylose (▪); arabinose (∘).

FIG. 3 shows a schematic representation of the setup of SBR I. New cycles of batch cultivation were initiated by either manual or automated replacement of approximately 90% of the culture with synthetic medium containing either 20 g l⁻¹ glucose, or 20 g l⁻¹ xylose and 20 g l⁻¹ arabinose.

FIG. 4 shows a CO₂ production profile (solid line) repeated batch cultivation (SBR I) in MY containing 20 g l⁻¹ xylose and 20 g l⁻¹ arabinose. The empty-fill regime was interrupted by filling the reactor with MY containing 20 g l⁻¹ glucose on two occasions, after batch 4 and 6. For all the batches, the specific growth rate calculated from the CO₂ production ().

FIG. 5 shows overlayed CO₂ production profiles of the repeated batches during SBR run I in MY containing 20 g l⁻¹ xylose and 20 g l⁻¹. Batch 2 (solid grey line); batch 4, 8, 12 (dotted lines); batch 16 (solid black line).

FIG. 6 shows a schematic representation of the setup of SBR II. New cycles of batch cultivation were initiated by either manual or automated replacement of approximately 90% of the culture with synthetic medium containing either 20 g l⁻¹ glucose, 20 g l⁻¹ xylose and 20 g l⁻¹ arabinose, or 20 g l⁻¹ xylose and 20 g l⁻¹ arabinose, or 20 g l⁻¹ arabinose.

FIG. 7 shows the typical CO₂ production profile of one single cycle of repeated batch cultivation in MY containing 20 g l⁻¹ glucose, 20 g l⁻¹ xylose and 20 g l⁻¹ arabinose, or 20 g l⁻¹ xylose and 20 g l⁻¹ arabinose, or 20 g l⁻¹ arabinose.

FIG. 8 shows the specific growth rate during SBR II in MY containing 20 g l⁻¹ glucose, 20 g l⁻¹ xylose and 20 g l⁻¹ arabinose (circle), or 20 g l⁻¹ xylose and 20 g l⁻¹ arabinose (square), or 20 g l⁻¹ arabinose (triangle).

FIG. 9 shows overlayed CO₂ production profiles of the repeated batches during SBR run II in MY containing 20 g l⁻¹ glucose, 20 g l⁻¹ xylose and 20 g l⁻¹ arabinose (A), or 20 g l⁻¹ xylose and 20 g l⁻¹ arabinose (B), or 20 g l⁻¹ arabinose (C). Batch cycle 1 (solid grey line); batch cycle 7 (dotted line); batch cycle 13 (striped line); batch cycle 20 (solid black line).

FIG. 10 shows anaerobic batch cultivations in MY containing a mixture of 30 g l⁻¹ glucose, 15 g l⁻¹ D-xylose, and 15 g l⁻¹ L-arabinose of strain IMS0010. Solid line, cumulative CO₂ production; glucose (); xylose (▪); arabinose (∘); ethanol (▴). The amount of ethanol produced was assumed to be equal to the measured cumulative production of CO₂ minus the CO₂ production that occurred due to biomass synthesis and the CO₂ associated with acetate formation.

### DETAILED DESCRIPTION OF THE INVENTION

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The present invention relates to a method for selecting a strain of an organism capable of consumption of a mixed substrate comprising two or more carbon sources. Typically, the method is used to identify a strain of the organism which shows improved consumption of the mixed substrate in comparison to the starting or reference strain of the organism to which the method is applied. That is to say, the method may be used to improve the performance of an existing strain of an organism with respect to its ability to consume a mixed substrate, for example to select a strain of the organism which shows faster consumption of the carbon sources in the mixed substrate.

Typically, the method is used to select a strain of an organism which has improved consumption on a mixed substrate so that it shows improved fermentation characteristics. Thus, a strain of an organism which has been selected according to the invention may show improved performance in terms of increased productivity, for example on a volumetric basis, of the fermentation product in question. Also, or alternatively, a strain of an organism selected using the method of the invention may also show an increase in yield of the fermentation product (in comparison to the strain from which it was selected).

In the method of the invention, a population of the organism is grown, that is to say selected, in the presence of two or more carbon sources. If desired, the method may be carried out with three, four, five or more carbon sources.

Typically, each carbon source will be a product derived from the hydrolysis of a carbohydrate (polysaccharide), for example a hydrolysis product derived from starch, cellulose, hemicellulose, lignocellulose, pectin or a material containing such carbohydrates. Such carbon sources include oligosaccharides, disaccharides and monosaccharides. The latter two are referred to herein as sugars.

Monosaccharides which may be used in the invention include: a triose, for example an aldotriose such as glyceraldehyde or a ketotriose such as dihydroxyacetone; a tetrose, for example an aldotetrose such as erythrose or threose or a ketotetrose such as erythrulose; a pentose, for example an aldopentose such as: arabinose, lyxose, ribose or xylose, or a ketopentose such as ribulose or xylulose; a hexose, for example an aldohexose such as allose, altrose, galactose, glucose, gulose, idose, mannose or talose or a ketohexose such as fructose, psicose, sorbose or tagatose or a sugar acid such as galacturonic acid; a heptoses, for example a keto-heptose such as mannoheptulose or sedoheptulose; an octose, such as octolose or 2-keto-3-deoxy-manno-octonate; or a nonoses such as sialose.

Disaccharides which may be used in the invention include sucrose, lactose, maltose, trehalose, cellobiose, gentiobiose, isomaltose, kojibiose, laminaribiose, mannobiose, melibiose, nigerose, rutinose or xylobiose.

The invention may preferably be carried out using a combination of two or more monosaccharides, for example two, three, four, five or more monosaccharides. Preferably, the two or more monosaccharides will all be hexoses, or be pentoses or be a combination of those two types of monosaccharide. A preferred combination of sugars is a combination of xylose and arabinose or a combination of xylose, arabinose and glucose. These combination represent the predominant sugars that are released in the hydrolysis of lignocellulosic feedstocks.

Growth of the population of the organism on the desired carbon sources exerts selection pressure on the population. Thus, mutants in the population may be selected for with an increased maximum specific growth rate (μₘₐₓ) on the carbon sources. If the selection pressure is maintained, for example by sequentially transferring batch-wise grown cultures to new batches, eventually (mutant) cells with a higher specific growth rate will overgrow all other cells with a lower specific growth rate.

The process of growing the microorgnism may e.g. be operated in batch culture, as a fed batch fermentation with constant feed or a continuous fermentation. These modes of operation in the presence of one or more monosaccharide are described in more detail hereunder:

Growth on Single Carbon Source (Monosaccharide)

Exponential Growth in Batch Cultures

The definition of a generation here is a doubling of yeast biomass. The doubling of the amount of biomass can be described by Cx (biomass concentration) at given time to be given by the following equation:

Cx(t)=Cx(0)*e^{(μ}*^{t)} (eq. 1)

The doubling time (Td in hr) or generation time (Tg hr) can be derived from the is equation by substituting Cx(t)=2*Cx(0).

Td=LN(2)/μ (hr) (eq. 2)

Where μ=specific growth rate in gr biomass/gr biomass/hr or 1/hr).

The biomass growth rate can be measured by various means: The increase of biomass amount can be analyzed by determining the amount of cells per weight or volume unit of a culture using any of the following method or a suitable alternative method:
Turbidity
Optical Density in the visible light spectrum (usual range: 600 nm to 700 nm) of a culture
A pellet volume after centrifugation,
The dry weight content after drying at constant weight at 105 C
Cell count per volume (microscopically),
Colony Forming Unit (CFU/ml) after plating on a solid agar medium and growing colonies on a plate from single cells

Alternatively one can derive the amount of biomass from a metabolic activity measured in a closed reactor system such as:
The rate of carbondioxide production (CPR carbondioxide production rate or CER Carbon Dioxide Evolution Rate generally expressed as mmol CO2/L/hr)
The rate of oxygen consumption (OUR Oxygen Uptake Rate mmol O2/L·hr)
Substrate uptake rate (rs=substrate uptake rate in g/L·hr uptake rate of glucose, xylose, arabinose or ammonia)

When Ln(Cx) or LN(CPR), LN(OUR) or LN (rs) or is plotted versus time in an exponential growth experiment (no nutrient limitations and no toxic products formed) a straight is obtained with the slope being the specific growth rate μ. With μ and eq. 2 one can calculate the doubling time and with the growth time one can calculate the amount of doublings or the number of generations.

Non Exponential Growth

In non-exponential growth experiments, e.g. a fed batch fermentation with constant feed or a continuous fermentation, the amount of generations is determined by calculating

Mx=Cx*Volume (biomass conc. in g/L*liter of broth produced in gr biomass) (eq 3.)

yielding the total mass of yeast biomass in gr dry matter of total CFU (=CFU/ml*ml of culture produced, or OD*vol.

A factor two increase in Mx means one generation.

The principal of the Non-exponential growth is also applicable to the exponential growth systems as described above.

Growth on Mixed Carbon Sources

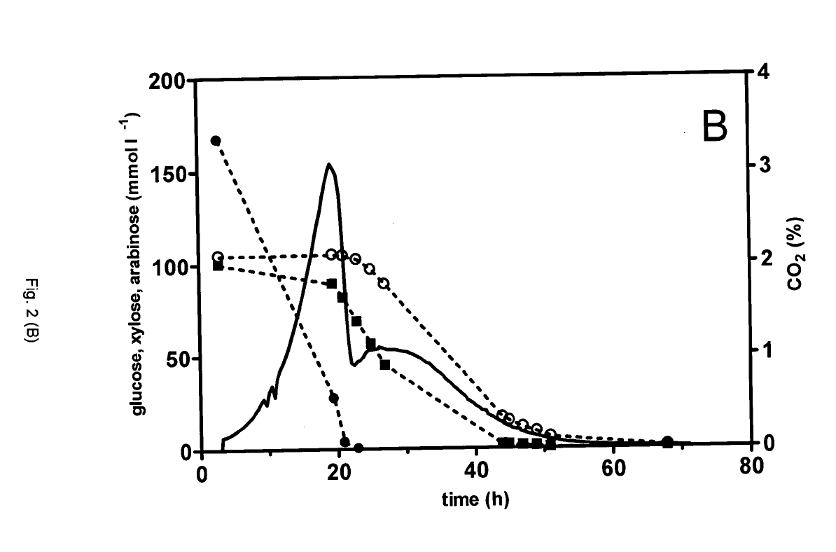

The system to describe and calculate the number of generations as described above for a single carbon source can also be applied to mixed substrates, e.g. mixes of glucose, xylose and arabinose. However to determine the number of generations on each of the individual substrates on has to correct the increase in total amount of biomass produces for the total amount of each of the individual sugar consumed. Therefore in these experiments one has to deduct which biomass increase corresponds to which sugar consumed. In table 2 an example is given for the calculation system on the basis of the assumption that first glucose is consumed, second xylose and third arabinose and which is true for less developed cases when the evolution is in it's initial stages as demonstrated in FIG. 2b.

To have always and exact calculation of the number of generations on a given substrate one could measure exactly the amount of each individual sugar consumed in each evolution experiment when sampled at very high frequency; eg. Every hr or every 2 hrs by making the balances over biomass increase (dMX/dt=total amount of biomass dry weight produced over the time interval) and substrate consumption (dMxyl=total amount of Xylose consumed in gr, dMara=total amount of arabinose consumed in gr or dMgluc=total amount of glucose consumed in gr). The generation fraction contributed to each of the individual sugar should then be calulcated over every doubling of Mx by the relative consumption of Xylose arabinose and glucose

e.g. number of generations on for a doubling of biomass (Mx) on xylose=the relative sugar consumption of xylose as compared to the overall sugar consumption or the sum of dMxyl, dMgluc and dMara=dS/dt or the total amount of sugar consumed over the same time interval of the specific doubling of the biomass.

dMxyl/((dMxyl+dMgluc+dMara). In this way one can exactly to determine the switch point from one substrate to the other in a batch experiment, which is relevant in the SBR set up on mixed substrates as described in this experiment but which is not so relevant e.g. in evolutions on mixed sugar concentrations in repeated fed batch or continuous cultivation systems under sugar limitation.

In the method of the invention, it is critical that the number of generations of growth of the population of the organism on each of the said carbon sources is at least about 50%, for example at least about 60%, such as at least about 70%, at least about 75%, at least about 80%, at least about 85%, at least about 90%, at least about 95% or at least 50%, for example at least 60%, such as at least 70%, at least 75%, at least 80%, at least 85%, at least 90%, at least 95%, at least 100%, at least 150%, at least 200% at least 250% or at least 300% of the number of generations of growth on the carbon source most preferred by the organism.

That is to say, the selection pressure to which the population is subjected in respect of each individual carbon source should be at least about half of that to which the population is subjected in relation to the most preferred carbon source. This will promote improvement of utilization of all of the carbon sources.

Accordingly, the number of generations of growth on each, and every, carbon source in the mixed substrate may be at least about 50%, at least about 60%, at least about, 70%, at least about 75%, at least about 80%, at least about 85%, at least about 90%, at least about 95% or at least about 98% of the number of generations of growth on the carbon source on which growth takes place for the greatest number of generations.

In the method of the invention, the least number of generations of growth typically takes place on the carbon source which is most preferred by the starting population of the organism. The next least number of generations of growth may then take place on the next most preferred carbon source, etc. Accordingly, the most number of generations of growth will typically take place on the carbon source which is least preferred by the organism.

The method of the invention may be carried out such that the number of generations of growth of the population of the organism on each carbon source is approximately equal. That is to say, the population of the organism is subjected to approximately equal selection pressure in relation to each and every carbon source.

Alternatively, the method may be carried out such that the number of generations of growth of the population of the organism on each carbon source is at least about equal to the number of generations of growth on the most preferred carbon source.

The selection pressure exerted on the population of the organism in relation to each or any carbon source may be increased by growing the population in the said each or any carbon source for a greater number of generations.

The terms “approximately equal” or “about equal” or the like in relation to numbers of generations of growth in the context of this invention is taken to indicate that the number of generations of growth in the presence of the carbon source on which the least number of generations of growth takes place is at least about 90%, such as at least about 95%, of the number of generations of growth in the presence of the carbon source on which the greatest number of generations of growth takes place.

For example, in the case of a yeast strain, such as a Saccharomyces cerevisiae strain, capable of utilizing xylose and arabinose which shows a preference for xylose over arabinose, the method may be carried out such that the number of generations of selection on arabinose is about the same or more than the number of generations of selection on xylose.

Typically, the method of the invention is carried out on an organism which consumes each of the two or more carbon sources sequentially.

The method of the invention may be carried out in any suitable format. However, the method may conveniently be carried out using a sequential batch reactor (SBR) protocol. In such a method, batch-wise grown cultures may be transferred sequentially to new batches. In such a method cells may be cultivated in repated batches by repeated, for example automated, replacement of the culture with fresh medium.

Typically, at least about 50%, at least about 60%, at least about 70%, at least abaout 80% or at least about 90% of the culture is replaced with fresh medium.

If the population of the organism is subjected to such a technique where all of the carbon sources are always present in the medium used to replenish the culture, the selection carried out will result in growth for a greater number of generations on the most preferred or more preferred carbon sources.

Accordingly, in the method of the invention, further selection is carried out such that additional generations of growth take place in the most preferred or more preferred carbon sources. This ensures that the number of generations of growth in the less preferred carbon source or sources is at least about 50% of the number of generations of growth in the most preferred carbon source. This protocol may be carried out so that the number of generations of growth in the less preferred carbon source or sources is at least about equal, or about equal, to the number of generations of growth in the most preferred carbon source

For example, in the case of two carbon sources, A and B, where A is more preferred than B, intitial selection may be carried out in the presence of A and B (during which selection the population will grow for more generations on A than B), followed by selection in B alone (during which selection the population of the organism will grow for a number of generations on B). This enables the number of generations of growth on B to about match or to exceed the number of generations of growth on A.

Where three carbon sources are used, A, B and C, where A is more preferred than B which is more preferred than C, selection may be carried out in the presence of A+B+C, followed by B+C, followed by C. This enables the number of generations of growth on B and C to about match or to exceed the number of generations of growth on A.

The method of the invention may be carried out in repeated cycles of selection. Thus, a method as described above with three carbon sources may be carried out with multiple cycles of selection, for example multiple cycles of A+B+C followed by B+C followed by C.

The method of the invention may be carried out using from about 5 to about 50 or more cycles of selection as described above, for example from about 10 to about 30 cycles of selection, such as about 20 cycles of selection.

In the method of the invention, the organism may undergo from about 10 to about 200 or more generations of growth on each carbon source, for example at least about 20, 30, 40, 50, 100, 150 or 200 or more generations of growth on each carbon source. Where multiple cycles of selection are used as described above, the number of generations of growth of the organism on each carbon source in each cycle may be at least about 3, 4, 5, 6, 7, 8, 9 or 10 or more.

The method is typically, carried out using selection on approximately equal concentrations of the carbon sources. That is to say, the concentrations of all of the carbon sources are within about 20%, such as about 10% for example about 5% of each other.

The concentration of a carbon source may be from about 10 gl⁻¹ to about 50 gl⁻¹ or more, for example about 20 gl⁻¹.

Selection in the invention will typically be carried out as a fermentation process.

Such a fermentation process may be an aerobic or an anaerobic fermentation process. An anaerobic fermentation process is herein defined as a fermentation process run in the absence of oxygen or in which substantially no oxygen is consumed, preferably less than about 5, about 2.5 or about 1 mmol/L/h, more preferably 0 mmol/L/h is consumed (i.e. oxygen consumption is not detectable), and wherein organic molecules serve as both electron donor and electron acceptors. In the absence of oxygen, NADH produced in glycolysis and biomass formation, cannot be oxidised by oxidative phosphorylation. To solve this problem many microorganisms use pyruvate or one of its derivatives as an electron and hydrogen acceptor thereby regenerating NAD⁺. Thus, in such a process in, pyruvate is used as an electron (and hydrogen) acceptor.

Alternatively, a method according to the invention may be carried out under oxygen limited conditions. Oxygen limited conditions may herein be defined as conditions wherein the dissolved oxygen concentration and/or oxygen availability is/are too low to sustain a completely respiratory mode of sugar metabolism thus leading to the use of pyruvate as an additional electron (and hydrogen) acceptor.

The method of the invention may in principle be carried out using any organism which may be cultured. Thus, an organism suitable for selection in the method of the invention may be a prokaryotic organism, for example a bacterium, or a eukaryotic cell, for example a yeast or a filamentous fungus. Herein, the term “cell” or “host cell” may be used to indicate an organism suitable for use in the method of the invention.

Yeasts are herein defined as eukaryotic microorganisms and include all species of the subdivision Eumycotina (Alexopoulos, C. J., 1962, In: Introductory Mycology, John Wiley & Sons, Inc., New York) that predominantly grow in unicellular form.

Yeasts may either grow by budding of a unicellular thallus or may grow by fission of the organism. A preferred yeast as a cell of the invention may belong to the genera Saccharomyces, Kluyveromyces, Candida, Pichia, Schizosaccharomyces, Hansenula, Kloeckera, Schwanniomyces or Yarrowia. Preferably the yeast is one capable of anaerobic fermentation, more preferably one capable of anaerobic alcoholic fermentation.

Filamentous fungi are herein defined as eukaryotic microorganisms that include all filamentous forms of the subdivision Eumycotina. These fungi are characterized by a vegetative mycelium composed of chitin, cellulose, and other complex polysaccharides.

The filamentous fungi of the kind suitable for use as a cell of the present invention are morphologically, physiologically, and genetically distinct from yeasts. Filamentous fungal cells may be advantageously used since most fungi do not require sterile conditions for propagation and are insensitive to bacteriophage infections. Vegetative growth by filamentous fungi is by hyphal elongation and carbon catabolism of most filamentous fungi is obligately aerobic.

Preferred filamentous fungi suitable for use in the method of the invention may belong to the genus Aspergillus, Trichoderma, Humicola, Acremoniurra, Fusarium or Penicillium. More preferably, the filamentous fungal cell may be a Aspergillus niger, Aspergillus oryzae, a Penicillium chrysogenum, or Rhizopus oryzae cell.

The invention may be used to select organisms which are capable of fermenting biomass, for example plant biomass, to a desired fermentation product, such as ethanol. Over the years suggestions have been made for the introduction of various organisms for the production of bio-ethanol from crop sugars. In practice, however, all major bio-ethanol production processes have continued to use the yeasts of the genus Saccharomyces as ethanol producer. This is due to the many attractive features of Saccharomyces species for industrial processes, i.e., a high acid-, ethanol- and osmo-tolerance, capability of anaerobic growth, and of course its high alcoholic fermentative capacity. Preferred yeast species as host cells include S. cerevisiae, S. bulderi, S. barnetti, S. exiguus, S. uvarum, S. diastaticus, K. lactis, K. marxianus or K. fragilis.

As set out above, the organism chosen for use in the method of the invention is typically one which is capable of fermenting the carbon sources to a desired product.

The fermentation product may be ethanol, butanol, lactic acid, 3-hydroxy-propionic acid, acrylic acid, acetic acid, succinic acid, citric acid, malic acid, fumaric acid, itaconic acid, an amino acid, 1,3-propane-diol, ethylene, glycerol, butanol, a β-lactam antibiotic or a cephalosporin.

The invention also relates to a strain of an organism identified or identifiable according to the method of the invention.

Typically, the method may be used to improve the performance of an organism, for example with respect to its ability to ferment carbon sources to a desired product.

The invention may preferentially be applied to a eukaryotic cell capable of expressing nucleotide sequences which confer on the cell the ability to use L-arabinose and/or to convert L-arabinose into L-ribulose, and/or xylulose 5-phosphate and/or into a desired fermentation product such as ethanol. These types of cells are described in detail in co-pending International patent application no. PCT/NL2007/000246.

Such cells express a nucleotide sequence encoding an arabinose isomerase (araA), a nucleotide sequence encoding a L-ribulokinase (araB), and a nucleotide sequence encoding an L-ribulose-5-P-4-epimerase (araD).

The nucleotide sequence encoding an araA may encode either a prokaryotic or an eukaryotic araA, i.e. an araA with an amino acid sequence that is identical to that of an araA that naturally occurs in the prokaryotic or eukaryotic organism. In co-pending International patent application no. PCT/NL2007/000246, a particular araA is described which confers on a host cell the ability to use arabinose and/or to convert arabinose into L-ribulose, and/or xylulose 5-phosphate and/or into a desired fermentation product such as ethanol when co-expressed with araB and araD. This does not depend so much on whether the araA is of prokaryotic or eukaryotic origin. Rather this depends on the relatedness of the araA's amino acid sequence to the specific sequence disclosed in SEQ ID NO. 1 of International patent application no. PCT/NL2007/000246 which is a Lactobacillus sequence.

The nucleotide sequence encoding an araB may encode either a prokaryotic or an eukaryotic araB, i.e. an araB with an amino acid sequence that is identical to that of a araB that naturally occurs in the prokaryotic or eukaryotic organism. In co-pending International patent application no. PCT/NL2007/000246, a particular araB is described which confers on a host cell the ability to use arabinose and/or to convert arabinose into L-ribulose, and/or xylulose 5-phosphate and/or into a desired fermentation product when co-expressed with araA and araD. This does not depend so much on whether the araB is of prokaryotic or eukaryotic origin. Rather this depends on the relatedness of the araB's amino acid sequence to the specific sequence disclosed in SEQ ID NO. 3 of co-pending International patent application no. PCT/NL2007/000246 which is a Lactobacillus sequence.

The nucleotide sequence encoding an araD may encode either a prokaryotic or an eukaryotic araD, i.e. an araD with an amino acid sequence that is identical to that of a araD that naturally occurs in the prokaryotic or eukaryotic organism. In co-pending International patent application no. PCT/NL2007/000246, a particular araD is described which confers on a host cell the ability to use arabinose and/or to convert arabinose into L-ribulose, and/or xylulose 5-phosphate and/or into a desired fermentation product when co-expressed with araA and araB. This does not depend so much on whether the araD is of prokaryotic or eukaryotic origin. Rather this depends on the relatedness of the araD's amino acid sequence to the specific sequence disclosed in SEQ ID NO. 5 of In co-pending International patent application no. PCT/NL2007/000246 which is a Lactobacillus sequence.

The codon bias index indicates that expression of the Lactobacillus plantarum araA, araB and araD genes were more favorable for expression in yeast than the prokaryolic araA, araB and araD genes described in EP 1 499 708.

L. plantarum is a Generally Regarded As Safe (GRAS) organism, which is recognized as safe by food registration authorities. Therefore, a preferred nucleotide sequence encodes an araA, araB or araD respectively having an amino acid sequence that is related to the sequences SEQ ID NO: 1, 3, or 5 respectively as defined in co-pending International patent application no. PCT/NL2007/000246. A preferred nucleotide sequence encodes a fungal araA, araB or araD respectively (e.g. from a Basidiomycete), more preferably an araA, araB or araD respectively from an anaerobic fungus, e.g. an anaerobic fungus that belongs to the families Neocallimastix, Caecomyces, Piromyces, Orpinomyces, or Ruminomyces. Alternatively, a preferred nucleotide sequence encodes a bacterial araA, araB or araD respectively, preferably from a Gram-positive bacterium, more preferably from the genus Lactobacillus, most preferably from Lactobacillus plantarum species. Preferably, one, two or three or the araA, araB and araD nucleotide sequences originate from a Lactobacillus genus, more preferably a Lactobacillus plantarum species. The bacterial araA expressed in a cell suitable for use in the invention may alternatively be the Bacillus subtilis araA disclosed in EP 1 499 708 and given as SEQ ID NO:9. SEQ ID NO:10 represents the nucleotide acid sequence coding for SEQ ID NO:9. The bacterial araB and araD expressed in the cell of the invention may alternatively be the ones of Escherichia coli (E. coli) as disclosed in EP 1 499 708 and given as SEQ ID NO: 11 and SEQ ID NO:13. SEQ ID NO: 12 represents the nucleotide acid sequence coding for SEQ ID NO:11. SEQ ID NO:14 represents the nucleotide acid sequence coding for SEQ ID NO:13.

To increase the likelihood that the (bacterial) araA, araB and araD enzymes respectively are expressed in active form in a eukaryotic host cell such as yeast, the corresponding encoding nucleotide sequence may be adapted to optimise its codon usage to that of the chosen eukaryotic host cell (Wiedemann and Boles Appl. Environ. Microbiol. 2008; 0: AEM.02395-07v1—electronic publication ahead of print). The adaptiveness of a nucleotide sequence encoding the araA, araB, and araD enzymes (or other enzymes of the invention, see below) to the codon usage of the chosen host cell may be expressed as codon adaptation index (CAI). The codon adaptation index is herein defined as a measurement of the relative adaptiveness of the codon usage of a gene towards the codon usage of highly expressed genes. The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Jansen et al., 2003, Nucleic Acids Res. 31(8):2242-51). An adapted nucleotide sequence preferably has a CAI of at least 0.2, 0.3, 0.4, 0.5, 0.6 or 0.7.

Expression of the nucleotide sequences encoding an araA, an araB and an araD confers to the cell the ability to use L-arabinose and/or to convert it into L-ribulose, and/or xylulose 5-phosphate. Without wishing to be bound by any theory, L-arabinose is expected to be first converted into L-ribulose, which is subsequently converted into xylulose 5-phosphate which is the main molecule entering the pentose phosphate pathway. In the context of the invention, “using L-arabinose” preferably means that the optical density measured at 660 nm (OD₆₆₀) of transformed cells cultured under aerobic or anaerobic conditions in the presence of at least 0.5% L-arabinose during at least 20 days is increased from approximately 0.5 till 1.0 or more. More preferably, the OD₆₆₀ is increased from 0.5 till 1.5 or more. More preferably, the cells are cultured in the presence of at least 1%, at least 1.5%, at least 2% L-arabinose. Most preferably, the cells are cultured in the presence of approximately 2% L-arabinose.

Typically, a cell is able “to convert L-arabinose into L-ribulose” when detectable amounts of L-ribulose are detected in cells cultured under aerobic or anaerobic conditions in the presence of L-arabinose (same preferred concentrations as in previous paragraph) during at least 20 days using a suitable assay. Preferably the assay is HPLC for L-ribulose.

Typically, a cell is able “to convert L-arabinose into xylulose 5-phosphate” when an increase of at least 2% of xylulose 5-phosphate is detected in cells cultured under aerobic or anaerobic conditions in the presence of L-arabinose (same preferred concentrations as in previous paragraph) during at least 20 days using a suitable assay. Preferably, an HPCL-based assay for xylulose 5-phosphate has been described in Zaldivar J., et al ((2002), Appl. Microbiol. Biotechnol., 59:436-442). This assay is briefly described in the experimental part. More preferably, the increase is of at least 5%, 10%, 15%, 20%, 25% or more.

Expression of the nucleotide sequences encoding an araA, araB and araD as defined earlier herein may also confer on the cell the ability to convert L-arabinose into a desired fermentation product when cultured under aerobic or anaerobic conditions in the presence of L-arabinose (same preferred concentrations as in previous paragraph) during at least one month till one year. More preferably, a cell is able to convert L-arabinose into a desired fermentation product when detectable amounts of a desired fermentation product are detected using a suitable assay and when the cells are cultured under the conditions given in previous sentence. Even more preferably, the assay is HPLC. Even more preferably, the fermentation product is ethanol.

A cell for transformation with the nucleotide sequences encoding the araA, araB, and araD enzymes respectively as described above, preferably is a host cell capable of active or passive xylose transport into and xylose isomerisation within the cell. The cell preferably is capable of active glycolysis. The cell may further contain an endogenous pentose phosphate pathway and may contain endogenous xylulose kinase activity so that xylulose isomerised from xylose may be metabolised to pyruvate.

The cell further preferably contains enzymes for conversion of pyruvate to a desired fermentation product such as ethanol, lactic acid, 3-hydroxy-propionic acid, acrylic acid, acetic acid, succinic acid, citric acid, malic acid, fumaric acid, itaconic acid, an amino acid, 1,3-propane-diol, ethylene, glycerol, butanol, a β-lactam antibiotic or a cephalosporin. The cell may be made capable of producing butanol by introduction of one or more genes of the butanol pathway as disclosed in WO2007/041269.

A host cell that has been transformed with a nucleic acid construct comprising the nucleotide sequence encoding the araA, araB, and araD enzymes as defined above. Such a the host cell may be co-transformed with three nucleic acid constructs, each nucleic acid construct comprising the nucleotide sequence encoding araA, araB or araD. The nucleic acid construct comprising the araA, araB, and/or araD coding sequence is capable of expression of the araA, araB, and/or araD enzymes in the host cell. To this end the nucleic acid construct may be constructed as described in e.g. WO 03/0624430. The host cell may comprise a single copy but preferably comprises multiple copies of each nucleic acid construct. The nucleic acid construct may be maintained episomally and thus comprise a sequence for autonomous replication, such as an ARS sequence. Suitable episomal nucleic acid constructs may e.g. be based on the yeast 2μ or pKD1 (Fleer et al., 1991, Biotechnology 9:968-975) plasmids. Preferably, however, each nucleic acid construct is integrated in one or more copies into the genome of the host cell. Integration into the host cell's genome may occur at random by illegitimate recombination but preferably nucleic acid construct is integrated into the host cell's genome by homologous recombination as is well known in the art of fungal molecular genetics (see e.g. WO 90/14423, EP-A-0 481 008, EP-A-0 635 574 and U.S. Pat. No. 6,265,186).

Accordingly, a cell suitable for use in the selection method of the invention may comprise a nucleic acid construct comprising the araA, araB, and/or araD coding sequence and is capable of expression of the araA, araB, and/or araD gene products. In an even more preferred embodiment, the araA, araB, and/or araD coding sequences are each operably linked to a promoter that causes sufficient expression of the corresponding nucleotide sequences in a cell to confer to the cell the ability to use L-arabinose, and/or to convert L-arabinose into L-ribulose, and/or xylulose 5-phosphate. Preferably the cell is a yeast cell. Accordingly, in a further aspect, the invention also encompasses a nucleic acid construct as earlier outlined herein. Preferably, a nucleic acid construct comprises a nucleic acid sequence encoding an araA, araB and/or araD. Nucleic acid sequences encoding an araA, araB, or araD have been all earlier defined herein.

Even more preferably, the expression of the corresponding nucleotide sequences in a cell confer to the cell the ability to convert L-arabinose into a desired fermentation product as defined later herein. In an even more preferred embodiment, the fermentation product is ethanol. Even more preferably, the cell is a yeast cell.

As used herein, the term “operably linked” refers to a linkage of polynucleotide elements (or coding sequences or nucleic acid sequence) in a functional relationship. A nucleic acid sequence is “operably linked” when it is placed into a functional relationship with another nucleic acid sequence. For instance, a promoter or enhancer is operably linked to a coding sequence if it affects the transcription of the coding sequence. Operably linked means that the nucleic acid sequences being linked are typically contiguous and, where necessary join two protein coding regions, contiguous and in reading frame.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most environmental and developmental conditions. An “inducible” promoter is a promoter that is active under environmental or developmental regulation.

The promoter that could be used to achieve the expression of the nucleotide sequences coding for araA, araB and/or araD may be not native to the nucleotide sequence coding for the enzyme to be expressed, i.e. a promoter that is heterologous to the nucleotide sequence (coding sequence) to which it is operably linked. Although the promoter preferably is heterologous to the coding sequence to which it is operably linked, it is also preferred that the promoter is homologous, i.e. endogenous to the host cell. Preferably the heterologous promoter (to the nucleotide sequence) is capable of producing a higher steady state level of the transcript comprising the coding sequence (or is capable of producing more transcript molecules, i.e. mRNA molecules, per unit of time) than is the promoter that is native to the coding sequence, preferably under conditions where arabinose, or arabinose and glucose, or xylose and arabinose or xylose and arabinose and glucose are available as carbon sources, more preferably as major carbon sources (i.e. more than 50% of the available carbon source consists of arabinose, or arabinose and glucose, or xylose and arabinose or xylose and arabinose and glucose), most preferably as sole carbon sources. Suitable promoters in this context include both constitutive and inducible natural promoters as well as engineered promoters. A preferred promoter for use in the present invention will in addition be insensitive to catabolite (glucose) repression and/or will preferably not require arabinose and/or xylose for induction.

Promotors having these characteristics are widely available and known to the skilled person. Suitable examples of such promoters include e.g. promoters from glycolytic genes, such as the phosphofructokinase (PFK), triose phosphate isomerase (TPI), glyceraldehyde-3-phosphate dehydrogenase (GPD, TDH3 or GAPDH), pyruvate kinase (PYK), phosphoglycerate kinase (PGK) promoters from yeasts or filamentous fungi; more details about such promoters from yeast may be found in (WO 93/03159). Other useful promoters are ribosomal protein encoding gene promoters, the lactase gene promoter (LAC4), alcohol dehydrogenase promoters (ADH 1, ADH4, and the like), the enolase promoter (ENO), the glucose-6-phosphate isomerase promoter (PGI1, Hauf et al, 2000) or the hexose(glucose) transporter promoter (HXT7) or the glyceraldehyde-3-phosphate dehydrogenase (TDH3). The sequence of the PGI1 promoter is given in SEQ ID NO:51. The sequence of the HXT7 promoter is given in SEQ ID NO:52. The sequence of the TDH3 promoter is given in SEQ ID NO:49. Other promoters, both constitutive and inducible, and enhancers or upstream activating sequences will be known to those of skill in the art. The promoters used in the host cells of the invention may be modified, if desired, to affect their control characteristics.

A preferred cell of the invention is a eukaryotic cell transformed with the araA, araB and araD genes of L. plantarum. More preferably, the eukaryotic cell is a yeast cell, even more preferably a S. cerevisiae strain transformed with the araA, araB and araD genes of L. plantarum. Most preferably, the cell is either CBS 120327 or CBS 120328 both deposited at the CBS Institute (The Netherlands) on Sep. 27, 2006.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically be operably linked to another promoter sequence or, if applicable, another secretory signal sequence and/or terminator sequence than in its natural environment. When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as earlier presented. Preferably the region of identity is greater than about 5 bp, more preferably the region of identity is greater than 10 bp.

The term “heterologous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced, but has been obtained from another cell or synthetically or recombinantly produced. Generally, though not necessarily, such nucleic acids encode proteins that are not normally produced by the cell in which the DNA is transcribed or expressed. Similarly exogenous RNA encodes for proteins not normally expressed in the cell in which the exogenous RNA is present. Heterologous nucleic acids and proteins may also be referred to as foreign nucleic acids or proteins. Any nucleic acid or protein that one of skill in the art would recognize as heterologous or foreign to the cell in which it is expressed is herein encompassed by the term heterologous nucleic acid or protein. The term heterologous also applies to non-natural combinations of nucleic acid or amino acid sequences, i.e. combinations where at least two of the combined sequences are foreign with respect to each other.

A cell suitable for use in the selection method of the invention that expresses araA, araB and araD is able to use L-arabinose and/or to convert it into L-ribulose, and/or xylulose 5-phosphate and/or a desired fermentation product as earlier defined herein and additionally exhibits the ability to use xylose and/or convert xylose into xylulose. The conversion of xylose into xylulose is preferably a one step isomerisation step (direct isomerisation of xylose into xylulose). This type of cell is therefore able to use both L-arabinose and xylose. “Using” xylose has preferably the same meaning as “using” L-arabinose as earlier defined herein.

Enzyme definitions are as used in WO 06/009434, for xylose isomerase (EC 5.3.1.5), xylulose kinase (EC 2.7.1.17), ribulose 5-phosphate epimerase (5.1.3.1), ribulose 5-phosphate isomerase (EC 5.3.1.6), transketolase (EC 2.2.1.1), transaldolase (EC 2.2.1.2), and aldose reductase” (EC 1.1.1.21).

Preferably, a cell suitable for use the selection method of the invention expressing araA, araB and araD as earlier defined herein has the ability of isomerising xylose to xylulose as e.g. described in WO 03/0624430 or in WO 06/009434. The ability of isomerising xylose to xylulose is conferred to the host cell by transformation of the host cell with a nucleic acid construct comprising a nucleotide sequence encoding a xylose isomerase. The transformed host cell's ability to isomerise xylose into xylulose is the direct isomerisation of xylose to xylulose. This is understood to mean that xylose isomerised into xylulose in a single reaction catalysed by a xylose isomerase, as opposed to the two step conversion of xylose into xylulose via a xylitol intermediate as catalysed by xylose reductase and xylitol dehydrogenase, respectively.

The nucleotide sequence encodes a xylose isomerase that is preferably expressed in active form in the transformed host cell of the invention. Thus, expression of the nucleotide sequence in the host cell produces a xylose isomerase with a specific activity of at least about 0.5 U xylose isomerase activity per mg protein at 30° C., preferably at least about 1, 2, 5, 10, 20, 25, 30, 50, 100, 200, 300 or 500 U per mg at 30° C. The specific activity of the xylose isomerase expressed in the transformed host cell is herein defined as the amount of xylose isomerase activity units per mg protein of cell free lysate of the host cell, e.g. a yeast cell free lysate. A unit (U) of xylose isomerise activity is herein defined as the amount of enzyme producing 1 nmol of xylulose per minute, under conditions as described by Kuyper et al. (2003, FEMS Yeast Res. 4, 69-78).

Preferably, expression of the nucleotide sequence encoding the xylose isomerase in the host cell produces a xylose isomerase with a Kₘ for xylose that is less than 50, 40, 30 or 25 mM, more preferably, the Kₘ for xylose is about 20 mM or less.

The nucleotide sequence encoding the xylose isomerase may encode either a prokaryotic or an eukaryotic xylose isomerase, i.e. a xylose isomerase with an amino acid sequence that is identical to that of a xylose isomerase that naturally occurs in the prokaryotic or eukaryotic organism. The present inventors have found that the ability of a particular xylose isomerase to confer to a eukaryotic host cell the ability to isomerise xylose into xylulose does not depend so much on whether the isomerase is of prokaryotic or eukaryotic origin. Rather this depends on the relatedness of the isomerase's amino acid sequence to that of the Piromyces sequence (SEQ ID NO. 7 in co-pending International patent application no. PCT/NL2007/000246). Surprisingly, the eukaryotic Piromyces isomerase is more related to prokaryotic isomerases than to other known eukaryotic isomerases. Therefore, a preferred nucleotide sequence encodes a xylose isomerase having an amino acid sequence that is related to the Piromyces sequence as defined above. A preferred nucleotide sequence encodes a fungal xylose isomerase (e.g. from a Basidiomycete), more preferably a xylose isomerase from an anaerobic fungus, e.g. a xylose isomerase from an anaerobic fungus that belongs to the families Neocallimastix, Caecomyces, Piromyces, Orpinomyces, or Ruminomyces. Alternatively, a preferred nucleotide sequence encodes a bacterial xylose isomerase, preferably a Gram-negative bacterium, more preferably an isomerase from the class Bacteroides, or from the genus Bacteroides, most preferably from B. thetaiotaomicron (SEQ ID NO. 15).

To increase the likelihood that the xylose isomerase is expressed in active form in a eukaryotic host cell such as yeast, the nucleotide sequence encoding the xylose isomerase may be adapted to optimise its codon usage to that of the eukaryotic host cell as earlier defined herein.

A host cell suitable for use in the selection method of the invention and transformed with the nucleotide sequence encoding the xylose isomerase as described above, preferably is a host capable of active or passive xylose transport into the cell. The host cell preferably contains active glycolysis. The host cell may further contain an endogenous pentose phosphate pathway and may contain endogenous xylulose kinase activity so that xylulose isomerised from xylose may be metabolised to pyruvate.

The host further preferably contains enzymes for conversion of pyruvate to a desired fermentation product such as ethanol, lactic acid, 3-hydroxy-propionic acid, acrylic acid, acetic acid, succinic acid, citric acid, malic acid, fumaric acid, an amino acid, 1,3-propane-diol, ethylene, glycerol, butanol, a β-lactam antibiotic or a cephalosporin. A preferred host cell is a host cell that is naturally capable of alcoholic fermentation, preferably, anaerobic alcoholic fermentation. The host cell further preferably has a high tolerance to ethanol, a high tolerance to low pH (i.e. capable of growth at a pH lower than 5, 4, 3, or 2.5) and towards organic acids like lactic acid, acetic acid or formic acid and sugar degradation products such as furfural and hydroxy-methylfurfural, and a high tolerance to elevated temperatures. Any of these characteristics or activities of the host cell may be naturally present in the host cell or may be introduced or modified by genetic modification. A suitable cell is a eukaryotic microorganism like e.g. a fungus, however, most suitable as host cell are yeasts or filamentous fungi. Preferred yeasts and filamentous fungi have already been defined herein.

As used herein the wording host cell has the same meaning as cell. Also, the terms host cell and cell may be used interchangeably with the term organism.

The cell suitable for use in the selection method of the invention is preferably transformed with a nucleic acid construct comprising the nucleotide sequence encoding the xylose isomerase. The nucleic acid construct that is preferably used is the same as the one used comprising the nucleotide sequence encoding araA, araB or araD.

The cell suitable for use in the selection method of the invention which expresses araA, araB and araD, and exhibits the ability to directly isomerise xylose into xylulose, as earlier defined herein may further comprise a genetic modification that increases the flux of the pentose phosphate pathway, as described in WO 06/009434. In particular, the genetic modification causes an increased flux of the non-oxidative part pentose phosphate pathway. A genetic modification that causes an increased flux of the non-oxidative part of the pentose phosphate pathway is herein understood to mean a modification that increases the flux by at least a factor 1.1, 1.2, 1.5, 2, 5, 10 or 20 as compared to the flux in a strain which is genetically identical except for the genetic modification causing the increased flux. The flux of the non-oxidative part of the pentose phosphate pathway may be measured by growing the modified host on xylose as sole carbon source, determining the specific xylose consumption rate and substracting the specific xylitol production rate from the specific xylose consumption rate, if any xylitol is produced. However, the flux of the non-oxidative part of the pentose phosphate pathway is proportional with the growth rate on xylose as sole carbon source, preferably with the anaerobic growth rate on xylose as sole carbon source. There is a linear relation between the growth rate on xylose as sole carbon source (μₘₐₓ) and the flux of the non-oxidative part of the pentose phosphate pathway. The specific xylose consumption rate (Qₛ) is equal to the growth rate (μ) divided by the yield of biomass on sugar (Yₓₛ) because the yield of biomass on sugar is constant (under a given set of conditions: anaerobic, growth medium, pH, genetic background of the strain, etc.; i.e. Qₛ=μ/Yₓₛ). Therefore the increased flux of the non-oxidative part of the pentose phosphate pathway may be deduced from the increase in maximum growth rate under these conditions. In a preferred embodiment, the cell comprises a genetic modification that increases the flux of the pentose phosphate pathway.

Genetic modifications that increase the flux of the pentose phosphate pathway may be introduced in the host cell in various ways. These including e.g. achieving higher steady state activity levels of xylulose kinase and/or one or more of the enzymes of the non-oxidative part pentose phosphate pathway and/or a reduced steady state level of unspecific aldose reductase activity. These changes in steady state activity levels may be effected by selection of mutants (spontaneous or induced by chemicals or radiation) and/or by recombinant DNA technology e.g. by overexpression or inactivation, respectively, of genes encoding the enzymes or factors regulating these genes.

In a more preferred cell for use in the selection method of the invention, the genetic modification comprises overexpression of at least one enzyme of the (non-oxidative part) pentose phosphate pathway. Preferably the enzyme is selected from the group consisting of the enzymes encoding for ribulose-5-phosphate isomerase, ribulose-5-phosphate epimerase, transketolase and transaldolase, as described in WO 06/009434.

Various combinations of enzymes of the (non-oxidative part) pentose phosphate pathway may be overexpressed. E.g. the enzymes that are overexpressed may be at least the enzymes ribulose-5-phosphate isomerase and ribulose-5-phosphate epimerase; or at least the enzymes ribulose-5-phosphate isomerase and transketolase; or at least the enzymes ribulose-5-phosphate isomerase and transaldolase; or at least the enzymes ribulose-5-phosphate epimerase and transketolase; or at least the enzymes ribulose-5-phosphate epimerase and transaldolase; or at least the enzymes transketolase and transaldolase; or at least the enzymes ribulose-5-phosphate epimerase, transketolase and transaldolase; or at least the enzymes ribulose-5-phosphate isomerase, transketolase and transaldolase; or at least the enzymes ribulose-5-phosphate isomerase, ribulose-5-phosphate epimerase, and transaldolase; or at least the enzymes ribulose-5-phosphate isomerase, ribulose-5-phosphate epimerase, and transketolase. In one embodiment of the invention each of the enzymes ribulose-5-phosphate isomerase, ribulose-5-phosphate epimerase, transketolase and transaldolase are overexpressed in the host cell. More preferred is a host cell in which the genetic modification comprises at least overexpression of both the enzymes transketolase and transaldolase as such a host cell is already capable of anaerobic growth on xylose. In fact, under some conditions we have found that host cells overexpressing only the transketolase and the transaldolase already have the same anaerobic growth rate on xylose as do host cells that overexpress all four of the enzymes, i.e. the ribulose-5-phosphate isomerase, ribulose-5-phosphate epimerase, transketolase and transaldolase. Moreover, host cells overexpressing both of the enzymes ribulose-5-phosphate isomerase and ribulose-5-phosphate epimerase are preferred over host cells overexpressing only the isomerase or only the epimerase as overexpression of only one of these enzymes may produce metabolic imbalances.

There are various means available in the art for overexpression of enzymes in the cells suitable for use in the selection method of the invention. In particular, an enzyme may be overexpressed by increasing the copy number of the gene coding for the enzyme in the host cell, e.g. by integrating additional copies of the gene in the host cell's genome, by expressing the gene from an episomal multicopy expression vector or by introducing a episomal expression vector that comprises multiple copies of the gene.

Alternatively overexpression of enzymes in the host cells suitable for use in the method of the invention may be achieved by using a promoter that is not native to the sequence coding for the enzyme to be overexpressed, i.e. a promoter that is heterologous to the coding sequence to which it is operably linked. Suitable promoters to this end have already been defined herein.

The coding sequence used for overexpression of the enzymes preferably is homologous to the host cell suitable for use in the method of the invention. However, coding sequences that are heterologous to the host cell suitable for use in the method of the invention may likewise be applied, as mentioned in WO 06/009434.

A nucleotide sequence used for overexpression of ribulose-5-phosphate isomerase in the host cell suitable for use in the method of the invention is a nucleotide sequence encoding a polypeptide with ribulose-5-phosphate isomerase activity, whereby preferably the polypeptide has an amino acid sequence having at least 50, 60, 70, 80, 90 or 95% identity with SEQ ID NO. 17 or whereby the nucleotide sequence is capable of hybridising with the nucleotide sequence of SEQ ID NO. 18, under moderate conditions, preferably under stringent conditions.

A nucleotide sequence used for overexpression of ribulose-5-phosphate epimerase in the host cell suitable for use in the method of the invention is a nucleotide sequence encoding a polypeptide with ribulose-5-phosphate epimerase activity, whereby preferably the polypeptide has an amino acid sequence having at least 50, 60, 70, 80, 90 or 95% identity with SEQ ID NO. 19 or whereby the nucleotide sequence is capable of hybridising with the nucleotide sequence of SEQ ID NO. 20, under moderate conditions, preferably under stringent conditions.

A nucleotide sequence used for overexpression of transketolase in the host cell of the invention is a nucleotide sequence encoding a polypeptide with transketolase activity, whereby preferably the polypeptide has an amino acid sequence having at least 50, 60, 70, 80, 90 or 95% identity with SEQ ID NO. 21 or whereby the nucleotide sequence is capable of hybridising with the nucleotide sequence of SEQ ID NO. 22, under moderate conditions, preferably under stringent conditions.

A nucleotide sequence used for overexpression of transaldolase in the host cell of the invention is a nucleotide sequence encoding a polypeptide with transaldolase activity, whereby preferably the polypeptide has an amino acid sequence having at least 50, 60, 70, 80, 90 or 95% identity with SEQ ID NO. 23 or whereby the nucleotide sequence is capable of hybridising with the nucleotide sequence of SEQ ID NO. 24, under moderate conditions, preferably under stringent conditions.

Overexpression of an enzyme, when referring to the production of the enzyme in a genetically modified host cell, means that the enzyme is produced at a higher level of specific enzymatic activity as compared to the unmodified host cell under identical conditions. Usually this means that the enzymatically active protein (or proteins in case of multi-subunit enzymes) is produced in greater amounts, or rather at a higher steady state level as compared to the unmodified host cell under identical conditions. Similarly this usually means that the mRNA coding for the enzymatically active protein is produced in greater amounts, or again rather at a higher steady state level as compared to the unmodified host cell under identical conditions. Overexpression of an enzyme is thus preferably determined by measuring the level of the enzyme's specific activity in the host cell using appropriate enzyme assays as described herein. Alternatively, overexpression of the enzyme may be determined indirectly by quantifying the specific steady state level of enzyme protein, e.g. using antibodies specific for the enzyme, or by quantifying the specific steady level of the mRNA coding for the enzyme. The latter may particularly be suitable for enzymes of the pentose phosphate pathway for which enzymatic assays are not easily feasible as substrates for the enzymes are not commercially available. Preferably in the host cells of the invention, an enzyme to be overexpressed is overexpressed by at least a factor 1.1, 1.2, 1.5, 2, 5, 10 or 20 as compared to a strain which is genetically identical except for the genetic modification causing the overexpression. It is to be understood that these levels of overexpression may apply to the steady state level of the enzyme's activity, the steady state level of the enzyme's protein as well as to the steady state level of the transcript coding for the enzyme.

A cell suitable for use in the selection method of the invention and which expresses araA, araB and araD and exhibiting the ability to directly isomerise xylose into xylulose and optionally comprising a genetic modification that increase the flux of the pentose pathway as earlier defined herein may further comprise a genetic modification that increases the specific xylulose kinase activity. Preferably the genetic modification causes overexpression of a xylulose kinase, e.g. by overexpression of a nucleotide sequence encoding a xylulose kinase. The gene encoding the xylulose kinase may be endogenous to the host cell or may be a xylulose kinase that is heterologous to the host cell. A nucleotide sequence used for overexpression of xylulose kinase in the host cell suitable for use in the method of the invention is a nucleotide sequence encoding a polypeptide with xylulose kinase activity, whereby preferably the polypeptide has an amino acid sequence having at least 50, 60, 70, 80, 90 or 95% identity with SEQ ID NO. 25 or whereby the nucleotide sequence is capable of hybridising with the nucleotide sequence of SEQ ID NO. 26, under moderate conditions, preferably under stringent conditions.

A particularly preferred xylulose kinase is a xylulose kinase that is related to the xylulose kinase xylB from Piromyces as mentioned in WO 03/0624430. A more preferred nucleotide sequence for use in overexpression of xylulose kinase in the host cell suitable for use in the method of the invention is a nucleotide sequence encoding a polypeptide with xylulose kinase activity, whereby preferably the polypeptide has an amino acid sequence having at least 45, 50, 55, 60, 65, 70, 80, 90 or 95% identity with SEQ ID NO. 27 or whereby the nucleotide sequence is capable of hybridising with the nucleotide sequence of SEQ ID NO. 28, under moderate conditions, preferably under stringent conditions.

In the host cells of the invention, genetic modification that increases the specific xylulose kinase activity may be combined with any of the modifications increasing the flux of the pentose phosphate pathway as described above, but this combination is not essential for the invention. Thus, a host cell of the invention comprising a genetic modification that increases the specific xylulose kinase activity in addition to the expression of the araA, araB and araD enzymes as defined herein is specifically included in the invention. The various means available in the art for achieving and analysing overexpression of a xylulose kinase in the host cells of the invention are the same as described above for enzymes of the pentose phosphate pathway. Preferably in the host cells of the invention, a xylulose kinase to be overexpressed is overexpressed by at least a factor 1.1, 1.2, 1.5, 2, 5, 10 or 20 as compared to a strain which is genetically identical except for the genetic modification causing the overexpression. It is to be understood that these levels of overexpression may apply to the steady state level of the enzyme's activity, the steady state level of the enzyme's protein as well as to the steady state level of the transcript coding for the enzyme.

In a further preferred embodiment, a cell suitable for use in the selection method of the invention:
expressing araA, araB and araD, and exhibiting the ability to directly isomerise xylose into xylulose, and optionally
comprising a genetic modification that increase the flux of the pentose pathway and/or
further comprising a genetic modification that increases the specific xylulose kinase activity all as earlier defined herein
may further comprise a genetic modification that reduces unspecific aldose reductase activity in the host cell. Preferably, unspecific aldose reductase activity is reduced in the host cell by one or more genetic modifications that reduce the expression of or inactivate a gene encoding an unspecific aldose reductase, as described in WO 06/009434. Preferably, the genetic modifications reduce or inactivate the expression of each endogenous copy of a gene encoding an unspecific aldose reductase in the host cell. Host cells may comprise multiple copies of genes encoding unspecific aldose reductases as a result of di-, poly- or aneu-ploidy, and/or the host cell may contain several different (iso)enzymes with aldose reductase activity that differ in amino acid sequence and that are each encoded by a different gene. Also in such instances preferably the expression of each gene that encodes an unspecific aldose reductase is reduced or inactivated. Preferably, the gene is inactivated by deletion of at least part of the gene or by disruption of the gene, whereby in this context the term gene also includes any non-coding sequence up- or down-stream of the coding sequence, the (partial) deletion or inactivation of which results in a reduction of expression of unspecific aldose reductase activity in the host cell. A nucleotide sequence encoding an aldose reductase whose activity is to be reduced in the host cell of the invention is a nucleotide sequence encoding a polypeptide with aldose reductase activity, whereby preferably the polypeptide has an amino acid sequence having at least 50, 60, 70, 80, 90 or 95% identity with SEQ ID NO. 29 or whereby the nucleotide sequence is capable of hybridising with the nucleotide sequence of SEQ ID NO. 30 under moderate conditions, preferably under stringent conditions.

In a cell suitable for use in the invention, the expression of the araA, araB and araD enzymes as defined herein is combined with genetic modification that reduces unspecific aldose reductase activity. The genetic modification leading to the reduction of unspecific aldose reductase activity may be combined with any of the modifications increasing the flux of the pentose phosphate pathway and/or with any of the modifications increasing the specific xylulose kinase activity in the host cells as described above, but these combinations are not essential for the invention. Thus, a host cell expressing araA, araB, and araD, comprising an additional genetic modification that reduces unspecific aldose reductase activity is specifically included in the invention.

In a preferred embodiment, a cell suitable for use in the selection method of the invention is CBS 120327 deposited at the CBS (Centraalbureau voor Schimmelcultures, Uppsalalaan 8, 3584 CT Utrecht, The Netherlands) on Sep. 27, 2006, CBS 120328 deposited at the CBS on Sep. 27, 2006, CBS 121879 deposited at the CBS on Sep. 20, 2007 or CBS 122700 deposited at the CBS on 11 Mar. 2008. All of these strains were deposited by Delft University of Technology. The former three strains are described in co-pending International patent application no. PCT/NL2007/000246. The latter strain is described in detail in the Examples. All of the deposited strains are Saccharomyces cerevisiae strains that have been engineered so that they can consume arabinose and xylose.

All of the cells described above may be used to select strains which show improved properties in relation to xylose and/or arabinose utilisation.

The selection process of the invention may be continued as long as necessary. This selection process is preferably carried out for from about one week to about one year. However, the selection process may be carried out for a longer period of time if necessary.

During the selection process, the cells are preferably cultured in the presence of approximately 20 g/l L-arabinose and/or approximately 20 g/l xylose. The strain obtained at the end of this selection process is expected to be improved as to its capacities of using L-arabinose and/or xylose, and/or converting L-arabinose into L-ribulose and/or xylulose 5-phosphate and/or a desired fermentation product such as ethanol.

In this context “improved cell” or “improved organism” may mean that the obtained cell is able to use the carbon sources on which it is selected, such as L-arabinose and/or xylose, in a more efficient way than the cell it derives from. For example, the obtained cell is expected to grow better (increase of the specific growth rate of at least 2% than the cell it derives from under the same conditions) or consume the carbon sources more rapidly. Preferably, such increases are of at least about 4%, 6%, 8%, 10%, 15%, 20%, 25% or more. The specific growth rate may be calculated from OD₆₆₀ as known to the skilled person. Therefore, by monitoring the OD₆₆₀, one can deduce the specific growth rate.

In this context “improved cell” may also mean that the obtained cell converts the carbon sources on which it has been selected, such as L-arabinose into L-ribulose and/or xylulose 5-phosphate and/or a desired fermentation product such as ethanol, in a more efficient way than the cell it derives from. For example, the obtained cell is expected to produce higher amounts of a conversion product or fermentation product such as L-ribulose and/or xylulose 5-phosphate and/or a desired fermentation product such as ethanol: increase of at least one of these compounds of at least 2% than the cell it derives from under the same conditions. Preferably, the increase is of at least 4%, 6%, 8%, 10%, 15%, 20%, 25% or more. In this context “improved cell” or “improved organism” may also mean that the obtained cell converts xylose into xylulose and/or a desired fermentation product such as ethanol in a more efficient way than the cell it derives from. For example, the obtained cell/organism is expected to produce higher amounts of xylulose and/or a desired fermentation product such as ethanol: increase of at least one of these compounds of at least 2% than the cell it derives from under the same conditions. Preferably, the increase is of at least 4%, 6%, 8%, 10%, 15%, 20%, 25% or more.

In a strain of an organism selected using the method of the invention, at least one of the genetic modifications described above, modifications obtained by selection, may confer to the improved strain the ability to grow on L-arabinose and optionally xylose as carbon source, preferably as sole carbon source, and preferably under anaerobic conditions. Preferably the improved strain produces essentially no xylitol, e.g. the xylitol produced is below the detection limit or e.g. less than 5, 2, 1, 0.5, or 0.3% of the carbon consumed on a molar basis.

Preferably the improved strain has the ability to grow on L-arabinose and optionally xylose as sole carbon source at a rate of at least 0.001, 0.005, 0.01, 0.03, 0.05, 0.1, 0.2, 0.25 or 0.3 h⁻¹ under aerobic conditions, or, if applicable, at a rate of at least 0.001, 0.005, 0.01, 0.03, 0.05, 0.07, 0.08, 0.09, 0.1, 0.12, 0.15 or 0.2 h⁻¹ under anaerobic conditions. Preferably the improved has the ability to grow on a mixture of glucose and L-arabinose and optionally xylose (in a 1:1 weight ratio) as sole carbon source at a rate of at least 0.001, 0.005, 0.01, 0.03, 0.05, 0.1, 0.2, 0.25 or 0.3 h⁻¹ under aerobic conditions, or, if applicable, at a rate of at least 0.001, 0.005, 0.01, 0.03, 0.05, 0.1, 0.12, 0.15, or 0.2 h⁻¹ under anaerobic conditions.

Preferably, the improved strain has a specific L-arabinose and, preferably, xylose consumption rate of at least about 100, 150, 200, 250, 300, 346, 350, 400, 500, 600, 650, 700, 750, 800, 900 or 1000 mg/g cells/h. Preferably, the modified host cell has a yield of fermentation product (such as ethanol) on L-arabinose and, preferably, xylose that is at least 20, 25, 30, 35, 40, 45, 50, 55, 60, 70, 80, 85, 90, 95 or 98% of the host cell's yield of fermentation product (such as ethanol) on glucose. More preferably, the modified host cell's yield of fermentation product (such as ethanol) on L-arabinose and, preferably, xylose is equal to the host cell's yield of fermentation product (such as ethanol) on glucose. Likewise, the modified host cell's biomass yield on L-arabinose and, preferably, xylose is preferably at least 55, 60, 70, 80, 85, 90, 95 or 98% of the host cell's biomass yield on glucose. More preferably, the modified host cell's biomass yield on L-arabinose and, preferably, xylose is equal to the host cell's biomass yield on glucose. It is understood that in the comparison of yields on glucose and L-arabinose and, preferably, xylose both yields are compared under aerobic conditions or both under anaerobic conditions.

Using the selection method of the invention, an improved yeast (Saccharomyces cerevisiae) strain has been isolated and deposited at the CBS (Centraalbureau voor Schimmelcultures, Uppsalalaan 8, 3584 CT Utrecht, The Netherlands) on 11 Mar. 2008 with the accession number CBS 122701. The depositor was Delft University of Technology.

In a preferred embodiment, a cell selected according to the invention expresses one or more enzymes that confer to the cell the ability to produce at least one fermentation product selected from the group consisting of ethanol, lactic acid, 3-hydroxy-propionic acid, acrylic acid, acetic acid, succinic acid, citric acid, malic acid, fumaric acid, an amino acid, 1,3-propane-diol, ethylene, glycerol, butanol, a β-lactam antibiotic and a cephalosporin. In a more preferred embodiment, the host cell of the invention is a host cell for the production of ethanol. In another preferred embodiment, the invention relates to a transformed host cell for the production of fermentation products other than ethanol. Such non-ethanolic fermentation products include in principle any bulk or fine chemical that is producible by a eukaryotic microorganism such as a yeast or a filamentous fungus. Such fermentation products include e.g. lactic acid, 3-hydroxy-propionic acid, acrylic acid, acetic acid, succinic acid, citric acid, malic acid, fumaric acid, itaconic acid, an amino acid, 1,3-propane-diol, ethylene, glycerol, butanol, a β-lactam antibiotic and a cephalosporin. A preferred host cell of the invention for production of non-ethanolic fermentation products is a host cell that contains a genetic modification that results in decreased alcohol dehydrogenase activity and/or reduced pyruvate decarboxylase activity.

In a further aspect, the invention relates to fermentation processes in which a strain of an organism selected using the method of the invention is used for the fermentation of a mixed substrate comprising two or more carbon sources, for example a substrate comprising a source of L-arabinose and optionally a source of xylose.

Preferably, the source of L-arabinose and the source of xylose are L-arabinose and xylose. In addition, the carbon source in the fermentation medium may also comprise a source of glucose. The source of L-arabinose, xylose or glucose may be L-arabinose, xylose or glucose as such or may be any carbohydrate oligo- or polymer comprising L-arabinose, xylose or glucose units, such as e.g. lignocellulose, xylans, cellulose, starch, arabinan and the like. For release of xylose or glucose units from such carbohydrates, appropriate carbohydrases (such as xylanases, glucanases, amylases and the like) may be added to the fermentation medium or may be produced by the modified host cell. In the latter case the modified host cell may be genetically engineered to produce and excrete such carbohydrases. An additional advantage of using oligo- or polymeric sources of glucose is that it enables to maintain a low(er) concentration of free glucose during the fermentation, e.g. by using rate-limiting amounts of the carbohydrases. This, in turn, will prevent repression of systems required for metabolism and transport of non-glucose sugars such as xylose. In a preferred process the modified host cell ferments both the L-arabinose (optionally xylose) and glucose, preferably simultaneously in which case preferably a modified host cell is used which is insensitive to glucose repression to prevent diauxic growth. In addition to a source of L-arabinose, optionally xylose (and glucose) as carbon source, the fermentation medium will further comprise the appropriate ingredient required for growth of the modified host cell. Compositions of fermentation media for growth of microorganisms such as yeasts or filamentous fungi are well known in the art.

In a preferred process, there is provided a process for producing a fermentation product selected from the group consisting of ethanol, lactic acid, 3-hydroxy-propionic acid, acrylic acid, acetic acid, succinic acid, citric acid, malic acid, fumaric acid, an amino acid, 1,3-propane-diol, ethylene, glycerol, butanol, a β-lactam antibiotic and a cephalosporin whereby the process comprises the steps of:
(a) fermenting a medium containing a two or more carbon sources with a strain of an organism selected using the method of the invention, and optionally,
(b) recovering the fermentation product.

The fermentation process is a process for the production of a fermentation product such as e.g. ethanol, lactic acid, 3-hydroxy-propionic acid, acrylic acid, acetic acid, succinic acid, citric acid, malic acid, fumaric acid, an amino acid, 1,3-propane-diol, ethylene, glycerol, butanol, a β-lactam antibiotic, such as Penicillin G or Penicillin V and fermentative derivatives thereof, and/or a cephalosporin. The fermentation process may be an aerobic or an anaerobic fermentation process. An anaerobic fermentation process is herein defined as a fermentation process run in the absence of oxygen or in which substantially no oxygen is consumed, preferably less than 5, 2.5 or 1 mmol/L/h, more preferably 0 mmol/L/h is consumed (i.e. oxygen consumption is not detectable), and wherein organic molecules serve as both electron donor and electron acceptors. In the absence of oxygen, NADH produced in glycolysis and biomass formation, cannot be oxidised by oxidative phosphorylation. To solve this problem many microorganisms use pyruvate or one of its derivatives as an electron and hydrogen acceptor thereby regenerating NAD⁺. Thus, in a preferred anaerobic fermentation process pyruvate is used as an electron (and hydrogen acceptor) and is reduced to fermentation products such as ethanol, lactic acid, 3-hydroxy-propionic acid, acrylic acid, acetic acid, succinic acid, citric acid, malic acid, fumaric acid, an amino acid, 1,3-propane-diol, ethylene, glycerol, butanol, a β-lactam antibiotics and a cephalosporin. In a preferred embodiment, the fermentation process is anaerobic. An anaerobic process is advantageous since it is cheaper than aerobic processes: less special equipment is needed. Furthermore, anaerobic processes are expected to give a higher product yield than aerobic processes. Under aerobic conditions, usually the biomass yield is higher than under anaerobic conditions. As a consequence, usually under aerobic conditions, the expected product yield is lower than under anaerobic conditions.

In another preferred embodiment, the fermentation process is under oxygen-limited conditions. More preferably, the fermentation process is aerobic and under oxygen-limited conditions. An oxygen-limited fermentation process is a process in which the oxygen consumption is limited by the oxygen transfer from the gas to the liquid. The degree of oxygen limitation is determined by the amount and composition of the ingoing gasflow as well as the actual mixing/mass transfer properties of the fermentation equipment used. In a process under oxygen-limited conditions, the rate of oxygen consumption may be at least about 5.5, for example at least about 6 or at least about 7 mmol/L/h.

The fermentation process is preferably run at a temperature that is optimal for the modified cell. Thus, for most yeasts or fungal cells, the fermentation process is performed at a temperature which is lower than 42° C., preferably lower than 38° C. For yeast or filamentous fungal host cells, the fermentation process is preferably performed at a temperature which is lower than 35, 33, 30 or 28° C. and at a temperature which is higher than 20, 22, or 25° C.

A preferred process is a process for the production of ethanol, whereby the process comprises the steps of: (a) fermenting a medium containing two or more carbon sources, for example L-arabinose and optionally xylose with a strain of an organism selected using the method of the invention, whereby the host cell ferments the carbon sources to ethanol; and optionally, (b) recovery of the ethanol.

The fermentation medium may also comprise a source of glucose that is also fermented to ethanol. In a preferred embodiment, the fermentation process for the production of ethanol is anaerobic. Anaerobic has already been defined earlier herein. In another preferred embodiment, the fermentation process for the production of ethanol is aerobic. In another preferred embodiment, the fermentation process for the production of ethanol is under oxygen-limited conditions, more preferably aerobic and under oxygen-limited conditions. Oxygen-limited conditions have already been defined earlier herein.

In the process, the volumetric ethanol productivity is preferably at least 0.5, 1.0, 1.5, 2.0, 2.5, 3.0, 5.0 or 10.0 g ethanol per litre per hour. The ethanol yield on L-arabinose and optionally xylose and/or glucose in the process preferably is at least 20, 25, 30, 35, 40, 45, 50, 60, 70, 80, 90, 95 or 98%. The ethanol yield is herein defined as a percentage of the theoretical maximum yield (which, for glucose and L-arabinose and optionally xylose is 0.51 g. ethanol per g. glucose or xylose), although it may be expressed in absolute terms. Accordingly, the invention also relates to a yeast strain, such as a Saccharomyces cerevisiae strain, capable of fermenting a substrate comprising xylose and arabinose, and optionally glucose, giving rise to an ethanol yield of at least about 0.2 g g¹, at least about 0.3 g g¹ or at least about 0.4 g g¹ or more.

The following Examples describe the invention:

### EXAMPLES

### Materials and Methods

Strains and maintenance. The Saccharomyces cerevisiae strains used in this study are listed in Table 1. Culture samples either from shake flasks, chemostat or (sequential) batch cultivations were stocked by the addition of 30% (v/v) glycerol and were stored in 2 ml aliquots at −80° C.

Media and shake-flask cultivation. Shake flask cultivations were performed at 30° C. in synthetic medium (MY), containing 5 g l⁻¹ (NH₄)₂SO₄, 3 g l⁻¹ KH₂PO₄, 0.5 g l⁻¹ MgSO₄.7H₂O, 0.05 ml l⁻¹ silicon antifoam and trace elements (Verduyn, C., E. Postma, W. A. Scheffers, and J. P. Van Dijken. 1992. Effect of benzoic acid on metabolic fluxes in yeasts: a continuous-culture study on the regulation of respiration and alcoholic fermentation. Yeast 8:501-517). For the cultivation in shake flasks, the pH of the medium was adjusted to 6.0 with 2 M KOH prior to sterilization. After heat sterilization (121° C., 20 min), a filter-sterilized vitamin solution (Verduyn et al., 1992, supra) and appropriate carbon and energy source were added. Shake flask cultures were prepared by inoculating 100 ml medium containing the appropriate sugar in a 500-ml shake flask with a frozen stock culture, and incubated at 30° C. in an orbital shaker (200 rpm).

Solid MY plates containing 20 g l⁻¹ xylose (MYX) or 20 g l⁻¹ arabinose (MYA) were prepared by adding 1.5% of agar to the MY. Plates were incubated at 30° C. until growth was observed.

Chemostat cultivation. Anaerobic chemostat cultivation was carried out at 30° C. in 2-L laboratory fermenters (Applikon, Schiedam, The Netherlands) with a working volume of 1-L. The culture was perfomed in synthetic medium supplemented with 0.01 g l-1 ergosterol and 0.42 g l⁻¹ Tween 80 dissolved in ethanol (Andreasen, A. A. and T. J. Stier. 1953. Anaerobic nutrition of Saccharomyces cerevisiae. I. Ergosterol requirement for growth in a defined medium. J. Cell Physiol. 41:23-36; and Andreasen, A. A. and T. J. Stier. 1954. Anaerobic nutrition of Saccharomyces cerevisiae. II. Unsaturated fatty acid requirement for growth in a defined medium. J. Cell Physiol. 43:271-281), silicon antifoam and trace elements (Verduyn et al., 1992, supra), and 20 g l⁻¹ xylose and arabinose as carbon and energy source, and was maintained at pH 5.0 by automatic addition of 2 M KOH. Cultures were stirred at 800 rpm and sparged with 0.5 l min⁻¹ nitrogen gas (<10 ppm oxygen). To minimize diffusion of oxygen, fermenters were equipped with Norprene tubing (Cole Palmer Instrument company, Vernon Hills, USA). Dissolved oxygen was monitored with an oxygen electrode (Applisens, Schiedam, The Netherlands). After completion the batch-wise growth, chemostat cultivation was initiated by the addition of synthetic medium containing 20 g l⁻¹ xylose and arabinose to the fermenter at a fixed dilution rate. The working volume of the culture was kept constant using an effluent pump controlled by an electric level sensor.

Sequential batch cultivation. For anaerobic sequential batch cultivation (SBR) the same fermenter setup and medium composition as for chemostat cultivation was used. New cycles of batch cultivation were initiated by either manual or automated replacement of approximately 90% of the culture with synthetic medium containing the appropriate carbon and energy source. Filling of the fermenter to a working volume of 1 liter was achieved using a feed pump controlled by an electric level sensor. Upon depletion of the carbon and energy source, indicated by the CO₂ percentage dropping below 0.05% after the CO₂ production peak, a new cycle was initiated by either manual or automated replacement of approximately 90% of the culture with fresh synthetic medium containing the appropriate carbon and energy source. For each cycle, the maximum specific growth rate was estimated from the CO₂ profile.

Batch cultivation. To characterize single colony isolates selected from the long-term chemostat and sequential batch cultivations, anaerobic batch cultivations were performed in 1 liter of synthetic medium containing 30 g l⁻¹ glucose, 15 g l⁻¹ D-xylose and 15 g l⁻¹ L-arabinose, using a similar fermenter setup as for the chemostat and sequential batch cultivations. Cultures to inoculate the batch fermentation were grown in shake flasks containing MY supplemented with 20 g l⁻¹ arabinose.

Preparation of Single Colony Isolate Cultures. Culture Samples Either from the chemostat or sequential batch cultivations (SBR I and II) were diluted and spread on solid MY containing 20 g l⁻¹ L-arabinose and incubated at 30° C. until colonies appeared. Separate colonies were re-streaked twice on solid MY with 20 g l⁻¹ L-arabinose. Single colonies were cultivated at 30° C. in shake flasks containing 100 ml MY supplemented with 20 g l⁻¹ L-arabinose. Frozen stock cultures were prepared by the addition of sterile glycerol to 30% (v/v) in the stationary growth phase, and storage of 2 ml aliquots at −80° C.

Determination of biomass dry weight. Culture samples (10.0 ml) were filtered over pre-weighed nitrocellulose filters (pore size 0.45 μm; Gelman laboratory, Ann Arbor, USA). After filtration of the broth, the biomass was washed with demineralised water and dried in a microwave oven for 20 min at 360 W and weighed. Duplicate determinations varied by less than 1%.

Gas analysis. Exhaust gas from the anaerobic fermenter cultivations was cooled in a condensor (2° C.) and dried with a Permapure dryer type MD-110-48P-4 (Permapure, Toms River, USA). Oxygen and carbon dioxide concentrations were determined with a NGA 2000 analyzer (Rosemount Analytical, Orrville, USA). Exhaust gas flow rate and specific carbon dioxide production rates were determined as described previously (Van Urk, H., P. R. Mak, W. A. Scheffers, and J. P. Van Dijken. 1988. Metabolic responses of Saccharomyces cerevisiae CBS 8066 and Candida utilis CBS 621 upon transition from glucose limitation to glucose excess. Yeast 4:283-291; and Weusthuis, R. A., W. Visser, J. T. Pronk, W. A. Scheffers, and J. P. Van Dijken. 1994. Effects of oxygen limitation on sugar metabolism in yeasts—a continuous-culture study of the Kluyver effect. Microbiology 140:703-715). In calculating the cumulative carbon dioxide production, volume changes caused by withdrawing culture samples were taken into account.

Metabolite analysis. Glucose, xylose, arabinose, xylitol, organic acids, glycerol and ethanol were analysed by HPLC using a Waters Alliance 2690 HPLC (Waters, Milford, USA) supplied with a BioRad HPX 87H column (BioRad, Hercules, USA), a Waters 2410 refractive-index detector and a Waters 2487 UV detector. The column was eluted at 60° C. with 0.5 g l⁻¹ sulfuric acid at a flow rate of 0.6 ml min⁻¹.

Rate calculations. For calculation of the specific rates of arabinose consumption and ethanol production, the time-dependent arabinose and ethanol data was fitted with Boltzmann sigmoidal equations. For each time point, the specific arabinose consumption rate and ethanol production rate were calculated by dividing the derivative/slope of the fitted curves by the dry weight.

Carbon recovery. Carbon recoveries were calculated as carbon in products formed, divided by the total amount of sugar carbon consumed, and were based on a carbon content of biomass of 48%. To correct for ethanol evaporation during the fermentations, the amount of ethanol produced was assumed to be equal to the measured cumulative production of CO₂ minus the CO₂ production that occurred due to biomass synthesis (5.85 mmol CO₂ per gram biomass (Verduyn et al., 1990, supra) and the CO₂ associated with acetate formation. Rate calculations. For calculation of the specific rates of arabinose and xylose consumption, the time-dependent arabinose and xylose data was fitted with the sigmoidal equation:

$$y ( x ) = {A}_{2} + \frac{( {A}_{1} + {A}_{2} )}{1 + exp ( \frac{x - {x}_{0}}{B \cdot x - C} )}$$

### Where:

A₁=initial value (left horizontal asymptote)
A₂=final value (right horizontal asymptote)
x₀=center (point of inflection)
T=width (change in x corresponding to the most significant change in the y axis)
B and C=parameters making T time dependent.

For each time point, the specific sugar consumption rate was calculated by dividing the derivative/slope of the fitted curves by the dry weight.

### Example 1

### Selection by Chemostat Cultivation

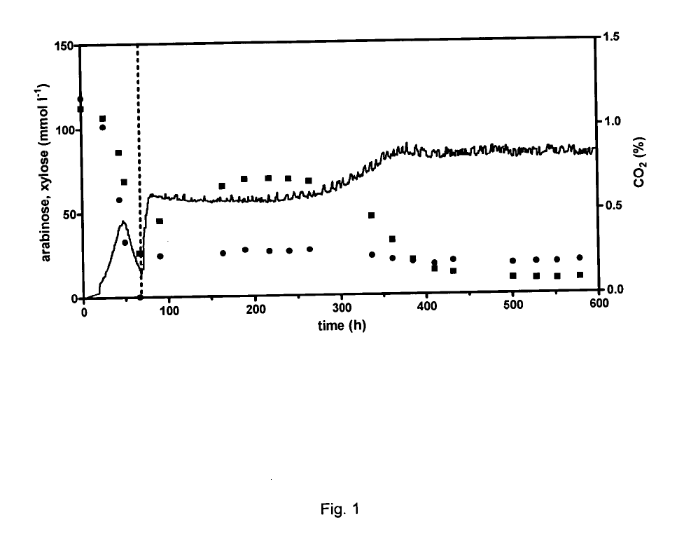

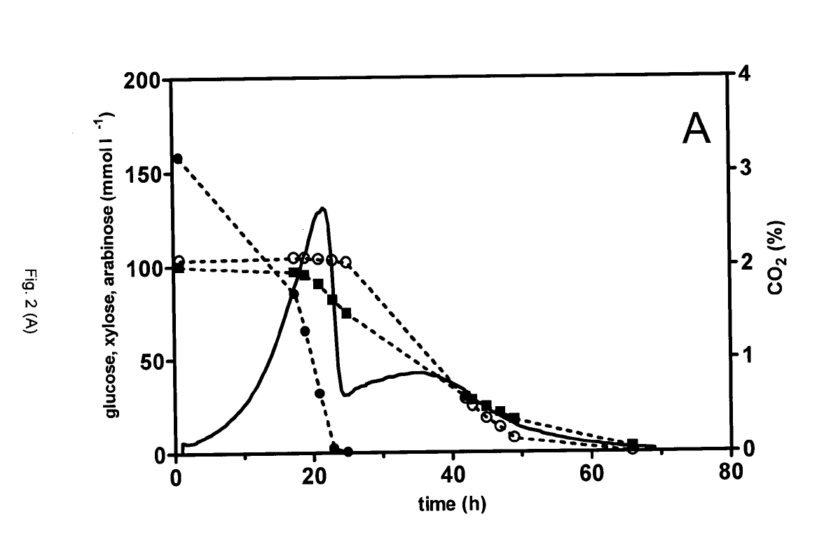

In co-pending International patent application number PCT/NL2007/000246, a xylose- and arabinose-fermenting S. cerevisiae strain IMS0003 (CBS 121879) was isolated after cultivation on solid MY with xylose and subsequent shake flask cultivation in MY supplemented with 20 g l⁻¹ arabinose. From a frozen stocks of this shake flask cultivation, a 100 mL shake flask culture was prepared by cultivation in MY containing 20 g l⁻¹ arabinose for 48 h at 30° C. and was used to inoculate an anaerobic fermenter containing 900 mL of MY with 20 g l⁻¹ xylose and 20 g l⁻¹ arabinose. After completion of the batch phase, chemostat cultivation was initiated by the addition of synthetic medium containing 20 g l⁻¹ xylose and 20 g l⁻¹ arabinose to the fermenter at a fixed dilution rate of 0.03 h⁻¹. During the chemostat cultivation, samples were withdrawn from the culture and biomass dry weight, xylose and arabinose concentrations were determined. Initially the xylose and arabinose concentration stabilized from 190 until approximately 250 hours at 69 and 26 mmol l-1 respectively (see FIG. 1). Between 250 and 600 hours of cultivation the residual xylose concentration in the continuous culture decreased from 69 mmol l⁻¹ to approximately 8.5 mmol l⁻¹, while the decrease of arabinose concentration was only minor, and remained at a level between 17 and 19 mmol l⁻¹. The results indicate that the affinity (defined as the μₘₐₓ/Kₛ) for xylose of the chemostat culture has increased, and that the affinity for arabinose did not change substantially.

Single colony isolates from the chemostat was tested for co-consumption of xylose and arabinose, by performing anaerobic batch fermentations containing a mixture of 30 g l⁻¹ glucose, 15 g l⁻¹ xylose and 15 g l⁻¹ arabinose. FIG. 2B shows the CO₂ production profile and the xylose and arabinose consumption during such a batch fermentation of one of the single colony isolates from the chemostat culture (strain IMS0007). The selective chemostat cultivation has resulted in a reduction of the total fermentation time of the glucose/xylose/arabinose mixture from approximately 70 hours (strain IMS0003, see FIG. 2A) to approximately 55 hours. With these results mainly indicating improved xylose consumption, the remaining challenge is improved co-consumption of xylose and arabinose.

IMS0007 has been deposited at the CBS (Centraalbureau voor Schimmelcultures, Uppsalalaan 8, 3584 CT Utrecht, The Netherlands) on 11 Mar. 2008 with the accession number CBS 122700. The depositor was Delft University of Technology.

### Example 2

### Selection by Sequential Batch Cultivation

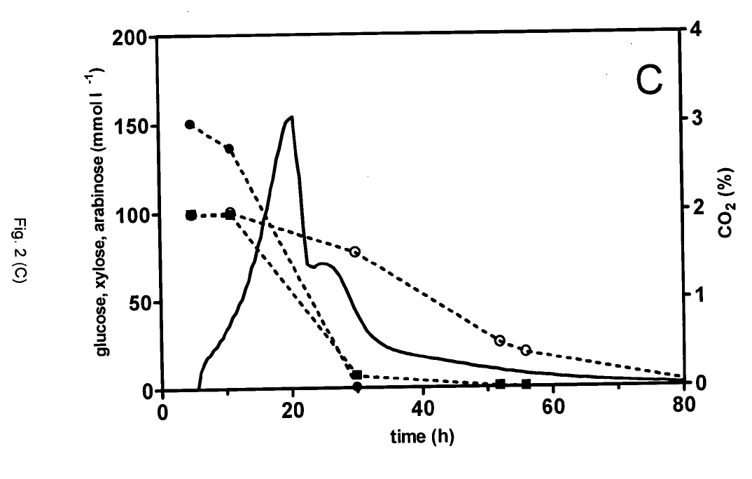

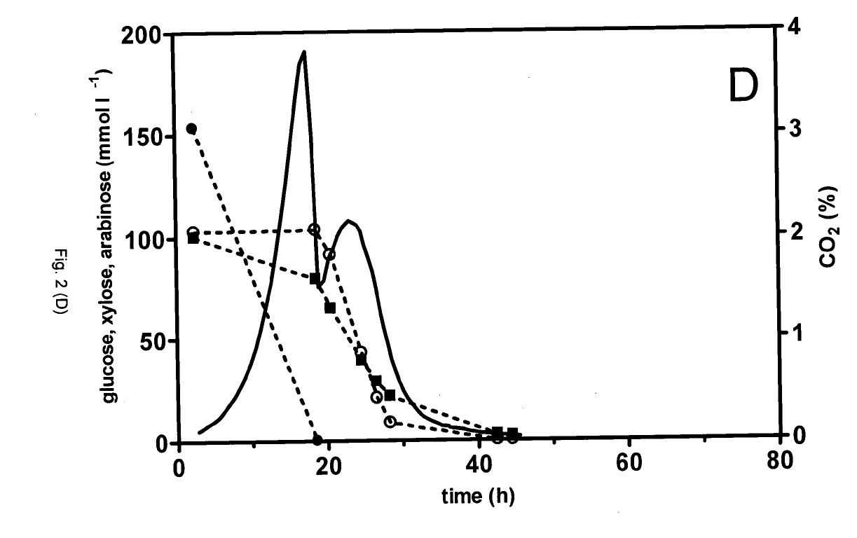

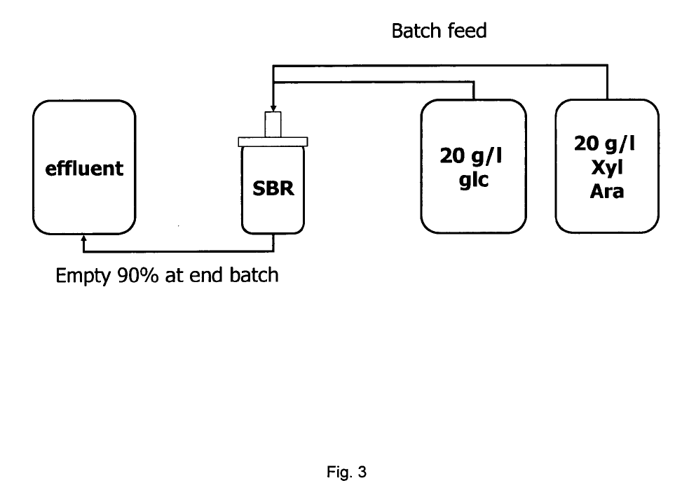

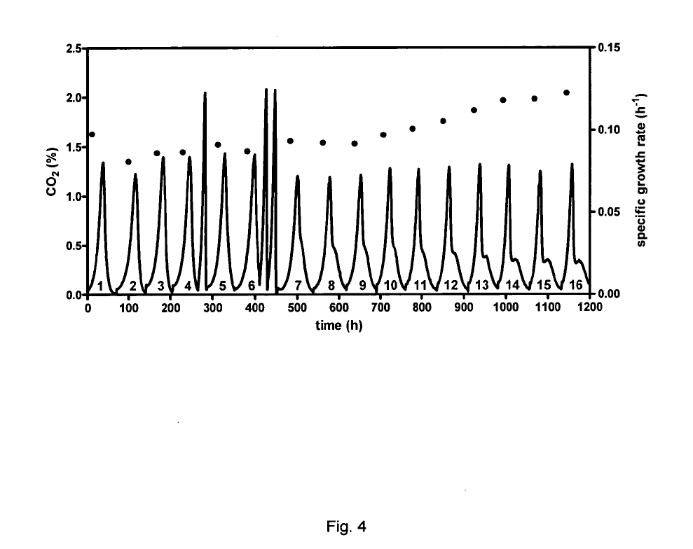

To obtain a S. cerevisiae strain with further improved xylose and arabinose co-consumption, compared to strain IMS0007, a sample from the chemostat selection cultivation was used to inoculate an anaerobic SBR fermenter. This system can be used for the selection of mutants with an increasing maximum specific growth rate (μmax). By sequentially transferring batch-wise grown cultures to new batches, eventually (mutant) cells with the highest specific growth rate will overgrow cells with a lower specific growth rate. In the first SBR run (SBR I), cells were cultivated in repeated batches by repeated automated replacement of approximately 90% of the culture with synthetic medium containing 20 g l⁻¹ xylose and 20 g l⁻¹ arabinose (FIG. 3). The first batch was initiated by inoculation of an anaerobic SBR containing 1 liter of this medium with a 100 ml culture sample from the chemostat selection cultivation as described above. Cells were cultivated in repeated batches using an automated fill-and-empty regime with MY containing 20 g l⁻¹ xylose and 20 g l⁻¹ arabinose. To select for cells with a constitutive phenotype of anaerobic co-consumption of xylose and arabinose, the regime was interrupted by filling the reactor with MY containing 20 g l⁻¹ glucose on two occasions, after batch 4 and 6 (see FIG. 4). For each cycle, the maximum specific growth rate was estimated from the CO₂ profile (see FIG. 4). After 16 cycles on medium supplemented with xylose and arabinose, the anaerobic specific growth rate increased from 0.08 to 0.13 h⁻¹. The carbon dioxide production profile and the deduced specific growth rates shows that the first phase of the batch cultivations on the xylose-arabinose mixture accelerated gradually during the course of the sequencing batch run. Analyses of sugars in supernatant samples showed that the observed acceleration was a result of increasing xylose consumption rates (data not shown). The arabinose consumption rates however, decreased during the SBR selection, resulting in a separation of the xylose and arabinose consumption represented by the two carbon dioxide production peaks rather than an improved co-consumption of xylose and arabinose. The overlays of the CO₂ production profiles of the repeated batches clearly show the shift from a single CO₂ production peak to the two-phased CO₂ production profile (see FIG. 5).

To compare the fermentation characteristics with the xylose- and arabinose fermenting strain IMS0007, a 100 mL sample was withdrawn from the SBR culture during batch 13 and used to inoculate an anaerobic batch fermenter containing MY supplemented with 30 g l⁻¹ glucose, 15 g l⁻¹ D-xylose, and 15 g l⁻¹ L-arabinose. The CO₂ production profile and sugar consumption plot of this anaerobic batch fermentation (see FIG. 2C) show that the xylose consumption had accelerated and the arabinose consumption was delayed compared to strain IMS0007 cultivated in MY medium containing the same sugar mixture (FIG. 2B).

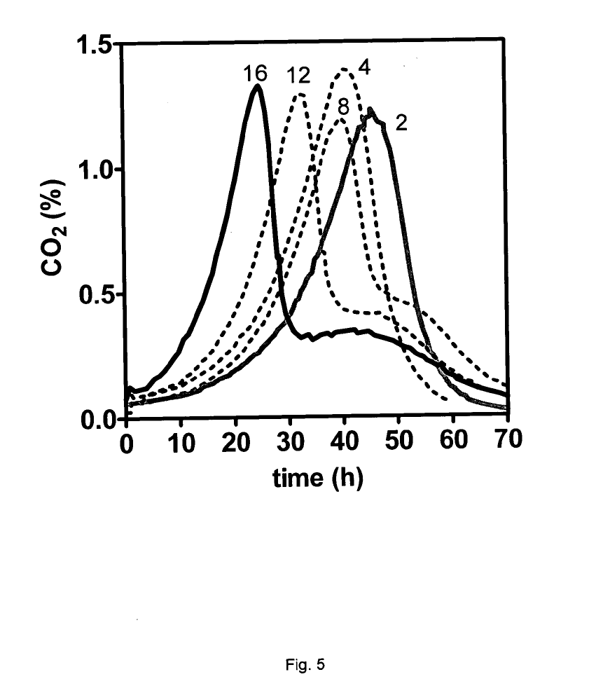

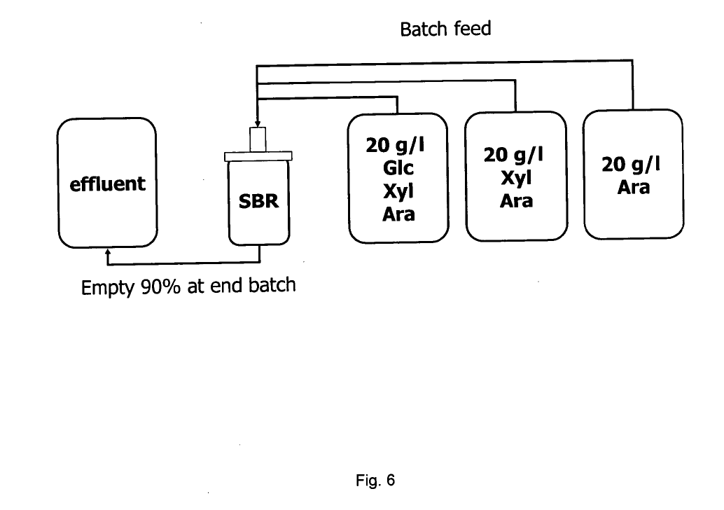

The observed shift during the SBR selection from co-consumption of xylose and arabinose to was probably due to the fact that the cells have a preference for xylose over arabinose, and as a consequence, the cells were grown for more generations on xylose compared to arabinose in the mixture of both sugars (see Table 2, 3.2 vs. 0.9 generations). To increase the selection pressure on the arabinose consumption, the number of generations of cells growing on arabinose should be increased. To accomplish this, a new SBR run (SBR II) was started. In SBR II, cells were cultivated in repeated batches by repeated automated replacement of approximately 90% of the culture with synthetic medium containing either 20 g l⁻¹ glucose, 20 g l⁻¹ xylose and 20 g l⁻¹ arabinose, or 20 g l⁻¹ xylose and 20 g l⁻¹ arabinose, or 20 g l⁻¹ arabinose (see FIG. 6). Table 2 indicates that in this setup, the number of generations on xylose and arabinose are in the same range, which should result in improvement of utilization of both sugars (4.2 vs. 4.6 generations).

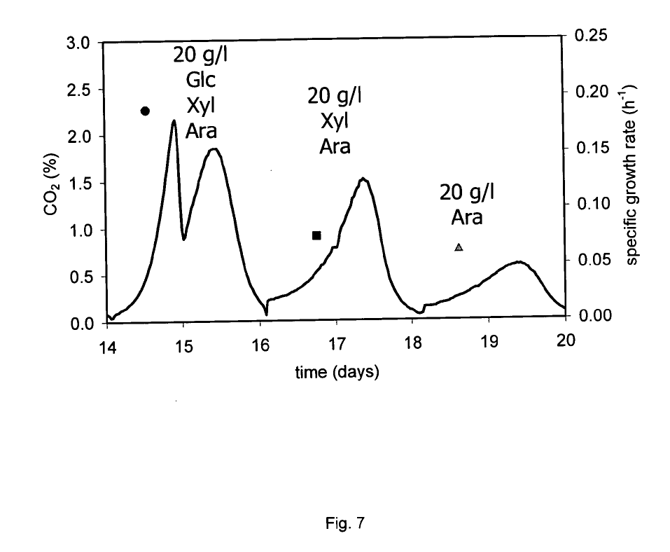

A single cycle of these 3 batch cultivations results in a typical CO₂ production profile as shown in FIG. 7. Cycles were repeated in this specific order for 20 times.

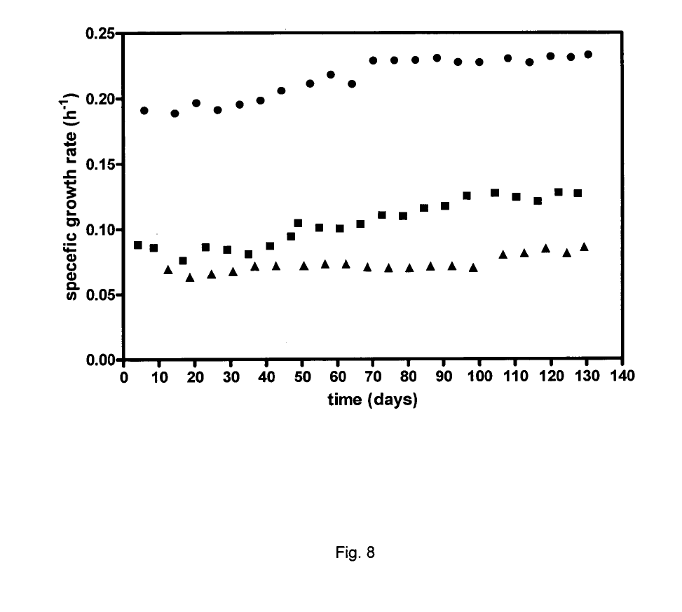

During the SBR II run the specific growth rates during the glucose/xylose/arabinose batches increased from 0.19 to approximately 0.23 h⁻¹ (FIG. 8). The growth rates during these batches were determined in the glucose consumption phase. Also the specific growth rate in the xylose/arabinose batches increased. However, the growth rate during the arabinose batches did not change.

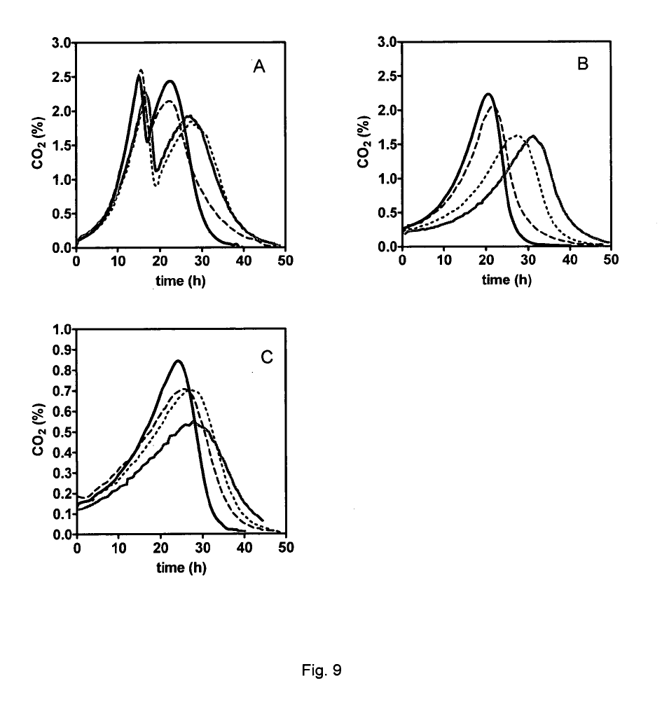

From the CO₂ production profiles of the separate batches (FIG. 9) could be deduced that, in contrast to SBR I, the capability to utilize xylose and arabinose simultaneously was preserved during SBR II. Moreover, the shape of the tail end of the CO₂ production peak shows an increased affinity for arabinose during the xylose/arabinose and arabinose batches. In addition, the total fermentation time of the sugar(s) in all the three batches decreased during the SBR run.

Single colony isolates from SBR II after approximately 3000 hours of cultivation were tested for their capability to co-consume xylose and arabinose. For this, arabinose grown re-streaked single colonies were cultivated anaerobically in MY containing a mixture of 30 g l⁻¹ glucose, 15 g l⁻¹ D-xylose, and 15 g l⁻¹ L-arabinose. The CO₂ production profile and the xylose and arabinose consumption during such a batch fermentation of one of the single colony isolates (strain IMS0010) are shown in FIG. 2D. The total fermentation time of the glucose/xylose/arabinose mixture was greatly reduced by the SBR selection to approximately 40 hours, compared to the previously selected strains IMS0003 and IMS0007. From the comparison of the sugar consumption profiles of strain IMS0010 with IMS0007 can be deduced that the arabinose utilization in particular has accelerated during the selection in SBR II, while the xylose consumption did not change substantially.

IMS0010 has been deposited at the CBS (Centraalbureau voor Schimmelcultures, Uppsalalaan 8, 3584 CT Utrecht, The Netherlands) on 11 Mar. 2008 with the accession number CBS 122701. The depositor was Delft University of Technology.

### Example 3

### Characterisation of the Strain IMS0010

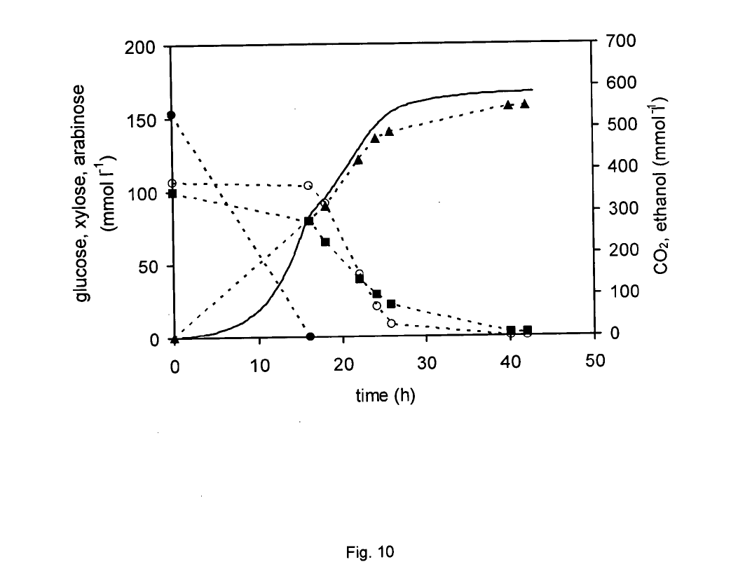

Strain IMS0010 was cultivated anaerobically in MY containing a mixture of 30 g l⁻¹ glucose, 15 g l⁻¹ D-xylose, and 15 g l⁻¹ L-arabinose. The cumulative CO₂ production profile, ethanol production and the xylose and arabinose consumption during such a batch fermentation are shown in FIG. 10. In this experiment, 153 mmol l⁻¹ glucose (27.6 g l⁻¹), 98 mmol l⁻¹ xylose (14.9 g l⁻¹) and 107 mmol l⁻¹ arabinose (16.0 g l⁻¹) were completely consumed within approximately 40 hours. The maximum specific consumption rates observed in this experiment were 0.49 g h⁻¹ (g dry weight)⁻¹ for arabinose, and 0.21 g h⁻¹ (g dry weight)⁻¹ for xylose. Estimated from the cumulative CO₂ production, 551 mmol l⁻¹ of ethanol (25 g l⁻¹) was produced, corresponding to an overall ethanol yield of 0.43 g g⁻¹ of total sugar. The total fermentation time of the glucose/xylose/arabinose mixture was greatly reduced by the SBR selection to approximately 40 hours, compared to the previously selected strains IMS0003 and IMS0007. From the comparison of the sugar consumption profiles of strain IMS0010 with IMS0007 can be deduced that the arabinose utilization in particular has accelerated during the selection in SBR II, while the xylose consumption did not change substantially.

To our knowledge, the above described strategy to improve the co-consumption of sequentially utilised sugars in sugar mixtures via SBR cultivation with an equal number of generations on each sugar, has not been described before. As a result we obtained cells with a higher specific growth rate, improved affinity and a reduction of the overall fermentation time.

TABLE 1

| Strains | Characteristics | Reference |
|---|---|---|
| IMS0003 | Single colony isolate of Strain IMS0002 cultivated anaerobically | PCT/NL2007/000246 |
| (CBS 121879) | on solid MY-xylose. Capable of co-fermenting mixtures of | |
| | glucose, xylose and arabinose to ethanol. | |
| IMS0007 | Single colony isolate strain obtained after long term chemostat | This work |
| (CBS 122700) | cultivation in MY supplemented with 20 g l⁻¹ xylose and 20 g l⁻¹ | |
| | arabinose. | |
| IMS0010 | Single colony isolate strain obtained after long term sequential | This work |
| (CBS 122701) | batch cultivation in MY supplemented with mixtures of glucose- | |
| | xylose-arabinose and xylose-arabinose, and arabinose as sole | |
| | carbon and energy source. | |

TABLE 2
Comparison of biomass formation of yeast cells cultivated in
an anaerobic batch fermentation in synthetic medium containing
different (mixtures of) carbon and energy source(s).

Increase in biomass (g l⁻¹) and number of
generations during batch on:

| Batch containing: | glucose | xylose | arabinose |
|---|---|---|---|
| 20 g l⁻¹ glucose + | 0.2 → 1.8 | 1.8 → 3.4 | 3.4 → 5.0 |
| 20 g l⁻¹ xylose + | (3.2) | (0.9) | (0.6) |
| 20 g l⁻¹ arabinose | | | |
| 20 g l⁻¹ xylose + | | 0.2 → 1.8 | 1.8 → 3.4 |
| 20 g l⁻¹ arabinose | | (3.2) | (0.9) |
| 20 g l⁻¹ arabinose | | | 0.2 → 1.8 |
| | | | (3.2) |
| Total nr. of | 3.2 | 4.2 | 4.6 |
| generations | | | |

Assumptions in this table: (i) glucose is the most preferred sugar, xylose is the second preferred sugar and arabinose is the least preferred sugar; (ii) in the mixtures of sugars, the sugars are consumed sequentially; (iii) the biomass yield is 0.08 g g⁻¹ of sugar.

1. A method for selecting a strain of an organism capable of improved consumption of a mixed substrate comprising two or more carbon sources as compared to a reference strain of the said organism, which method comprises:
growing a population of the reference strain of the organism in the presence of the two or more carbon sources, wherein the number of generations of growth of the said population on each of the said carbon sources is at least about 50% of the number of generations of growth on the carbon source most preferred by the reference strain of the organism; and
selecting the resulting strain of the organism,
thereby to select a strain of the organism capable of improved consumption of a mixed substrate comprising the two or more carbon sources as compared to the reference strain of the organism.

2. A method according to claim 1, wherein the number of generations of growth on each carbon source is approximately equal.

3. A method according to claim 1, wherein the number of generations of growth on each of the carbon sources is at least about 30.

4. A method according to claim 1, wherein the organism consumes each of the two or more carbon sources sequentially.

5. A method according to claim 1, wherein one or more of the carbon sources is a sugar.

6. A method according to claim 5, wherein one or more of the sugars is a monosaccharide or a disaccharide.

7. A method according to claim 6, wherein the monosaccharide is a hexose sugar or a pentose sugar.

8. A method according to claim 7, wherein the hexose sugar is allose, altrose, galactose, glucose, gulose, idose, mannose or talose.

9. A method according to claim 7, wherein the pentose sugar is arabinose, lyxose, ribose or xylose.

10. A method according to claim 1, wherein the organism is grown on a combination of carbon sources comprising xylose and arabinose.

11. A method according to claim 10, wherein the population of the organism is grown on a combination of carbon sources comprising glucose, xylose and arabinose.

12. A method according to claim 1, wherein the growth of the population of the organism is carried out by cultivation in sequential batch reactors (SBR).

13. A method according to claim 1, wherein the method is carried out under anaerobic conditions.

14. A method according to claim 1, wherein the method is carried out under aerobic conditions, preferably performed under oxygen limited conditions.

15. A method according to claim 1, wherein the organism is a eukaryotic organism.

16. A method according to claim 15, wherein the eukaryotic organism is a yeast.

17. A method according to claim 16, wherein the yeast is of the genus Saccharomyces, Kluyveromyces, Candida, Pichia, Schizosaccharomyces, Hansenula, Klockera, Schwanniomyces or Yarrowia.

18. A method according to claim 17, wherein the yeast is of the species S. cerevisiae, S. bulderi, S. bametti, S. exiguus, S. uvarum, S. diastaticus, K. lactis, K. marxianus or K. fragilis.

19. A method according to claim 1, wherein the eukaryotic organism is a filamentous fungus.

20. A method according to claim 19 wherein the filamentous fungus is of the genus Aspergillus, Penicillium, Rhizopus, Trichoderma, Humicola, Acremonium or Fusarium.

21. A method according to claim 20, wherein the filamentous fungus is of the species Aspergillus niger, Aspergillus oryzae, Penicillium chrysogenum, or Rhizopus oryzae.

22. A method according to claim 1, wherein the organism is capable of fermenting the carbon sources to a desired product.

23. A method according to claim 22, wherein the fermentation product is ethanol, butanol, lactic acid, 3-hydroxy-propionic acid, acrylic acid, acetic acid, succinic acid, citric acid, malic acid, fumaric acid, itaconic acid, an amino acid, 1,3-propane-diol, ethylene, glycerol, butanol, a β-lactam antibiotic and a cephalosporin.

24. A strain of an organism identified according to the method of claim 1.

25. A yeast strain capable of a specific consumption rate of arabinose of at least about 0.4 g h⁻¹ (g dry weight)⁻¹ and of xylose of at least about 0.2 g h⁻¹ (g dry weight)⁻¹.

26. A yeast strain capable of fermenting a substrate comprising xylose and arabinose, and optionally glucose, giving rise to an ethanol yield of at least about 0.4 g g⁻¹.

27. A Saccharomyces cerevisiae strain deposited at the Centraalbureau voor Schimmelcultures under the accession number CBS 122701.

28. A process for producing a fermentation product which process comprises fermenting a substrate containing two or more sources of carbon with a strain of an organism according to claim 24 such that the cell ferments the said carbon sources to the fermentation product.

29. A process according to claim 28, wherein the strain of the organism is one yeast strain capable of a specific consumption rate of arabinose of at least about 0.4 q h⁻¹ (q dry weight)⁻¹ and of xylose of at least about 0.2 q h⁻¹ (g dry weight)⁻¹ and the substrate comprises xylose and arabinose and optionally glucose.

30. A process for producing a fermentation product which process comprises: selecting a strain of an organism capable of consumption of a mixed substrate comprising two or more carbon sources using a method according to claim 1; and
fermenting a medium containing the two or more carbon sources on which the strain of the organism was selected with the strain of the organism thus selected such that the strain of the organism ferments the two or more carbon sources to the fermentation product.

31. A process according to claim 28, which process comprises recovering the fermentation product.

32. A process according to claim 28, wherein the fermentation product is ethanol, butanol, lactic acid, 3-hydroxy-propionic acid, acrylic acid, acetic acid, succinic acid, citric acid, malic acid, fumaric acid, itaconic acid, an amino acid, 1,3-propane-diol, ethylene, glycerol, butanol, a β-lactamantibiotic and a cephalosporin.

33. A process according to claim 28, wherein the process is anaerobic.

34. A process according to claim 28, wherein the process is aerobic, preferably performed under oxygen limited conditions.
